(19) United States
(12) Patent Application Publication
(10) Pub. No.: US 2026/0253203 A1
(43) Pub. Date: Aug. 27, 2026

(54) METHODS AND SYSTEMS FOR CHARACTERIZING POROSITY OF ADDITIVELY MANUFACTURED BONE SCAFFOLDS

(71) Applicant: MARSHALL UNIVERSITY RESEARCH CORPORATION, Huntington, WV (US)

(72) Inventors: Roozbeh Salary, Huntington, WV (US); Yousef Abdelgaber, Huntington, WV (US)

(21) Appl. No.: 19/134,310

(22) PCT Filed: Nov. 30, 2023
(86) PCT No.: PCT/US2023/081782

§ 371 Date: May 30, 2025

Related U.S. Application Data
(60) Provisional application No. 63/429,026, filed on Nov. 30, 2022.

(51) Int. Cl. G06T 7/00; A61F 2/00; G01N 15/08; G06T 7/13; G06T 7/136

(52) U.S. Cl. CPC G06T 7/001; G01N 15/088; G06T 7/13; G06T 7/136; A61F 2/0077; G06T 2207/30052; G06T 2207/30108

(57) ABSTRACT

A method for characterizing a bone tissue scaffold produced by an additive manufacturing process is provided. An image of the bone tissue scaffold is first captured and a boundary of the bone tissue scaffold is determined. One or more surface features of the bone tissue scaffold are subsequently detected using an edge detection algorithm, with the one or more surface features including one or more pores and/or one or more highlights. Edges that correspond to a pore using a pore filling algorithm are identified and the surface porosity of the bone tissue scaffold is thereby mapped. Systems for characterizing a bone tissue scaffold are also provided and make use of an image capture device for capturing an image of the bone tissue scaffold, as well as a processor for analyzing the image via instructions stored on the processor.

### RELATED APPLICATIONS

This application claims priority from U.S. Provisional Application Ser. No. 63/429,026, filed Nov. 30, 2022, the entire disclosure of which is incorporated herein by this reference.

### TECHNICAL FIELD

The presently-disclosed subject matter generally relates to methods and systems for characterizing the porosity of additively manufactured bone scaffolds. In particular, certain embodiments of the presently-disclosed subject matter relate to image-based methods and systems for characterizing pore size distribution and the dimensional properties of bone tissue scaffolds fabricated using additive manufacturing processes, such as pneumatic micro-extrusion.

### BACKGROUND

Osseous fractures and defects are significant contributors of disability in the world. Millions of patients suffer yearly from bone fractures and disorders, which are the most common causes of severe long-term pain as well as physical disabilities. The use of advanced manufacturing methods—such as pneumatic micro-extrusion (PME), selective laser sintering, and particulate leaching—for the fabrication of biocompatible scaffolds (capable of hosting bone morrow-derived stem cells) has emerged as a clinically effective as well as cost-effective approach. Most of the current treatment methods in clinical practice are centered on implantation of bone grafts and/or osteoconductive porous scaffolds, seeded with autologous stem cells for bone regeneration. Patient-specific treatment of bone fractures requires fabrication of mechanically robust, dimensionally accurate, and porous bone tissue scaffolds. Therefore, there is a need for characterization of scaffold porosity, morphology, and functional integrity during and after scaffold fabrication process.

The functional, mechanical, and medical properties of bone scaffolds are significantly influenced by porosity (as an intrinsic scaffold property). For example, porosity affects the rate of osteogenesis and ultimately fracture healing. It is expected that bone scaffolds are composed of interconnected pores, enhancing material transport. Too small pores prevent cell migration, limiting the diffusion of nutrients as well as removal of waste products. In addition, closed pores obstruct material transport (e.g. growth factors), cell proliferation, and osteoconduction inside a scaffold. Too large pores, while improving cellular infiltration, lead to a decrease in the surface area needed for cell adhesion and compromise the mechanical properties of scaffolds as a result of void volume. Also, sub-optimal pores adversely affect not only scaffold functional integrity but also osteoconduction as well as osseointegration. It has been shown that achieving mean pore sizes of 96-150 μm would facilitate optimal cell attachment, while large pores in the range of 300-800 μm would be required for successful bone growth in scaffolds. It has also been observed that optimal pore size depends on different cell types.

Detection and quantification of scaffold porosity aid in (i) a better understanding of cell activity/behavior and (ii) identification and prediction of the underlying phenomena influential on the functional performance (including failure), accuracy, and functional integrity of fabricated bone tissue scaffolds. Besides, if implemented in real-time during fabrication process, assessment of scaffold porosity allows for property control and thus achieving bone scaffolds with optimal functional and medical properties.

To date, however, the characterization of bone scaffold porosity and morphology has remained a complex image-processing problem since bone scaffolds (in an acquired image) are inherently composed of pores that are not discretely and completely bound (known as gradual pores). None of the current image processing methods can effectively address this problem without extensive use of, for example, X-ray computed tomography (CT) imaging, which cannot be implemented in situ nor practically in a common environment. Consequently, development of a robust image-based method that allows for in situ characterization of scaffold morphology with gradual pores would be both highly desirable and beneficial. In the absence of such a method, optimal fabrication as well as in situ characterization of bone scaffolds will remain trial-and-error-driven (not patient-specific) with no control over desired medical and functional properties.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

The presently-disclosed subject matter includes methods and systems for characterizing porosity of additively manufactured bone scaffolds. In particular, certain embodiments of the presently-disclosed subject matter include image-based methods and systems for characterizing pore size distribution and the dimensional properties of bone tissue scaffolds fabricated using additive manufacturing processes, such as pneumatic micro-extrusion.

In one exemplary implementation of a method for characterizing a bone tissue scaffold, a bone tissue scaffold is first provided where the bone tissue scaffold is produced by an additive manufacturing process. In some implementations, the additive manufacturing method used to produce the bone tissue scaffold is pneumatic micro-extrusion (PME). In some implementations, the materials used to produce the bone tissue scaffold comprise a polyester polymer, such as, in certain embodiments, polycaprolactone (PCL).

Once the bone tissue scaffold has been produced, as one of the initial steps in the process, an image of the bone tissue scaffold is obtained. In certain implementations, capturing the image of the bone tissue scaffold comprises capturing an image of the bone tissue scaffold using a high-resolution charge-coupled device (CCD) camera. In some such implementations, the captured image is a monochromatic image, such as a grayscale image.

Subsequent to obtaining the image of the bone tissue scaffold, a boundary of the bone tissue scaffold, as it appears in the image, is then obtained. In some implementations, the boundary of the bone tissue scaffold in the image is determined by initially positioning a bounding box around the bone tissue scaffold included in the image. The bounding box, in such implementations, is then incrementally shrunken while, during that shrinking process, an amount of material is discarded each time the bounding box is shrunk. To determine the appropriate boundary of the bone tissue scaffold in the image, a rate of change of discarded material is identified as the material is being discarded and once that rate of change is equal to or exceeds a predetermined threshold parameter, the boundary of the scaffold is then identified or otherwise determined. In implementations, the boundary of the bone tissue scaffold is determined in an iterative manner whereby after completing the foregoing boundary determination steps, the image is rotated, and the steps are repeated to thereby identify a boundary of the bone tissue scaffold with the least resulting area.

After the boundary to be used for further characterization of the bone tissue scaffold has been determined, one or more surface features of the bone tissue scaffold are subsequently detected through the use of an edge detection algorithm capable of detecting surface features, including one or more pores and one or more highlights (reflections) in the image. In some implementations, such an edge detection algorithm comprises a Canny edge detection algorithm. In some implementations, after detecting the various edges using an edge detection algorithm, an exemplary method of characterizing a bone tissue scaffold then further includes a step of identifying a continuous edge of the one or more detected surface features using a marching algorithm.

In some implementations, upon the identification of the edges of the surface features included in or shown in the image of the bone tissue scaffold, the edges corresponding to the one or more highlights are then filtered out to thereby identify the edges that correspond to the edges of a pore. In some implementations, to perform such a filtering step, a map is constructed using a plurality of image intensities from the image, and a gradient is calculated using the plurality of image intensities. From that mapping, edges corresponding to a pore are identified and are subsequently compared to the corresponding pore in the original image. The pore edges from the intensity mapping are processed using the using the edge detection algorithm, and an intersection of the pore edge from the mapping and the pore edge identified in the original image is further identified to map and characterize the pore. That process of pore characterization is then repeated for each pore included in the bone scaffold to thereby map the surface porosity of the produced bone tissue scaffold.

Further provided, in some embodiments of the presently-disclosed subject matter are systems for characterizing a bone tissue scaffold and which can be utilized to carry out the methods for characterizing a bone tissue scaffold in accordance with the presently-disclosed subject matter. In some embodiments, a system for characterizing a bone tissue scaffold is provided that comprises an image capture device for capturing an image of the bone tissue scaffold and a processor including instructions for analyzing the image. In some embodiments, the processor includes instructions for determining a boundary of the bone tissue scaffold shown in the image, detecting one or more surface features (e.g., pores or highlights), identifying edges that correspond to a pore shown in the image using a pore filling algorithm, and mapping the surface porosity of the bone tissue scaffold shown in the image.

Further features and advantages of the present invention will become evident to those of ordinary skill in the art after a study of the description, figures, and non-limiting examples in this document.

### BRIEF DESCRIPTION OF THE DRAWINGS

FIGS. 1A-1C includes images showing porous, biocompatible, and biodegradable bone scaffolds, allowing for cell incorporation and diffuse proliferation, where the scaffolds are composed of complex “gradual pores” that are characterized with intensity gradients and no discretely defined boundaries.

FIGS. 2A-2B include images showing (FIG. 2A) the components of the pneumatic micro extrusion (PME) process, and (FIG. 2B) polycaprolactone (PCL) deposition on a heated glass substrate (leading to bone scaffold formation), where the filtered air is introduced to the chamber at a constant rate, and which aids in obtaining a constant polymer solidification rate.

FIGS. 3A-3C include images showing different designs of bone scaffolds composed of PCL, where the bone scaffolds with different morphological characteristics were fabricated using the PME process.

FIG. 4 is a flowchart depicting an exemplary method of characterizing a bone tissue scaffold in accordance with the presently-disclosed method, and including noise filtering steps as well as a robust edge focused methodology, where edges from Canny edge detection are a building block, and where thresholding is not done until the pore filling block.

FIGS. 5A-5B include images showing (FIG. 5A) a monochromatic image of a fabricated porous bone scaffold, captured using a high-resolution CCD camera, and (FIG. 5B) image thresholding, image rotation, as well as scaffold bounding.

FIGS. 6A-6B include images showing (FIG. 6A) a PME-fabricated bone scaffold, composed of PCL, and (FIG. 6B) after Canny edge detection with an appropriate threshold, where, subsequently, the detected edges were color-coded after consolidation of unique edges and further refined by pore highlight (reflection) removal.

FIGS. 7A-7B include images showing a 3D height map (FIG. 7B) comparison of a gradual forming pore on the surface of a scaffold and its corresponding image reference (FIG. 7A), and illustrating that the challenge of identifying the pore is the lack of discrete border which defines where it begins and where it ends, as it is a gradually forming indentation.

FIGS. 8A-8B are schematic diagrams showing (FIG. 8A) a demonstration of the calculation method of gradients for each pixel, and (FIG. 8B) the resulting normalized gradient vectors of the detected edge.

FIGS. 9A-9C are images and diagrams demonstrating the process for discriminating pores from highlights, and showing a positive curvature (+curv.) and a negative curvature (−curv.), with image intensity representing absolute curvature intensity.

FIGS. 10A-10F are images illustrating the process of filling up a pore in the presence of noises such as highlights.

FIGS. 11A-11B are schematic diagrams illustrating the validation of the presently-disclosed methodology using varied simulated geometries, where colored regions correspond to detected pores, and where the algorithm identifies every surface-level pore in the rendering based off inspection of the colored regions.

FIGS. 12A-12B are images showing an exemplary approach to rendering a realistic benchmark, where the schwarz gyroid model design was loaded into a physically-based rendering software (Blender Cycles), and a simulated material was composed to match that of a glossy white PCL plastic, and where the complexity can be observed in the rendered output (FIG. 12A), containing only gradually forming pores, and highlights from reflected light.

FIG. 13 is a schematic diagram showing four different simulated lighting environments, with (a) a 50 W floor lamp, (b) a fluorescent ceiling lighting, (c) a flashlight shining 50 cm above, and (d) the 50 W floor lamp, but with a clay material, where the second column depicts pore filling image arrays produced by the presently-disclosed method with each consolidated pore is marked with a ‘+’ at the centroid with the color hue depicting relative size of pores, and where the final column depicts a comparison in pore filling to a standard methodology.

FIGS. 14A-14F includes images showing (FIG. 14A) a fabricated TPMS (Schwarz Gyroid-based) PCL bone scaffold, (FIG. 14B) the internal structure of a bone scaffold after cropping and gray scaling, (FIG. 14C) pore identification using the presently-disclosed method, and pore identification using a standard thresholding method (i.e., Otsu's method) at a threshold level of: (FIG. 14D) 0.50, (FIG. 14E) 0.70, and (FIG. 14F) 0.85 (showing that Otsu's method unlike the presently-disclosed method fails to function on the bone scaffold image and in general on multimodal images).

FIGS. 15A-15F includes images showing (FIG. 15A) Diamond geometry, (FIG. 15B) Schoen I-WP geometry, (FIG. 15C) Neovius geometry, where FIGS. 15A-15C show PME-fabricated bone scaffolds with a high, medium, and low level of pore quality, and where FIGS. 15D-15F show the production of the porosity surface mappings with regions showing larger individual pores, as well as relatively smaller pores.

FIG. 16A-16C includes graphs showing (FIG. 16A) the average percent relative standard deviation (RSD) of pore areas, (FIG. 16B) the average % RSD of pore pattern spacing distances, and (FIG. 16C) the average compression moduli found in a compression testing machine, where the error bars represent the standard deviation in measurements across the 3 sets of 6 samples for each of the metrics, and where the data in (FIG. 16A) and (FIG. 16B) was collected from a single photo (per scaffold) of the top layer of a printed scaffold in general lighting.

FIGS. 17A-17D include images and diagrams showing an exemplary workflow for analyzing pore geometry given a target STL image, including (FIG. 17A) an image depicting a cropped photo of a starch-based PME print, (FIG. 17B) an image depicting a cross-section image of the target geometry, (FIG. 17C) an image depicting the pores fitted optimally to the target geometry and shading depicting overflow (OF) and underflow (UF), and (FIG. 17D) an image depicting the final noise filtered results with centroid positions labeled with a marker, and color depicting relative size and showing larger pores and smaller pores.

FIG. 18 is a graph showing layer-by-layer quality metrics produced in real-time by the presently-disclosed method on a PME-fabricated hydroxyapatite-polysaccharide scaffold (depicted in FIG. 19), where the “RSD Area” represents the percent relative standard deviation (RSD) of pore areas, where “RSD Distance” represents the percent RSD of pore pattern spacing distance, and with the “Precision Error” being the weighted average of the two, where “Over-Under Flow %” represents the percent overflow or underflow when compared to target geometry, where “% Deviation STL” represents the absolute value of deviation, when compared to target geometry, and where the “Overall Error” is the an important metric and is a weighted average of all the previous metrics, and where notable is the improvement in quality as the scaffold walls begin to thin more and the pores begin to open at layer 11.

FIG. 19 includes images showing the deviation of the PME-fabricated hydroxyapatite-polysaccharide bone scaffold over time, where notable is the improvement in quality as the scaffold walls begin to thin more and the pores begin to open at layer 11, and where also notable is the complete closing of a pore by layer 29 (as a major defect) corresponding to the spiking of the error metrics (plotted in FIG. 18).

FIG. 20 is a schematic diagram showing a selected pixel in a chain with two possible options for the next pixel in the chain, where the arrow corresponds to the average direction of the previous few steps.

FIGS. 21A-21C include images showing (FIG. 21A) a surface of a bone scaffold, showing a highlight, (FIG. 22B) inaccurate highlight edge detection due to edge discontinuity, and (FIG. 21C) analysis of fill together with discrimination of edges, leading to accurate detection of the highlight's edges.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, databases, websites and other published materials referred to throughout the entire disclosure herein, unless noted otherwise, are incorporated by reference in their entirety.

Where reference is made to a URL or other such identifier or address, it is understood that such identifiers can change and particular information on the internet can come and go, but equivalent information can be found by searching the internet. Reference thereto evidences the availability and public dissemination of such information.

Although any methods, devices, and materials similar or equivalent to those described herein can be used in the practice or testing of the presently-disclosed subject matter, representative methods, devices, and materials are described herein.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a pore” includes a plurality of such pores, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

Bone tissue engineering has emerged as a promising strategy for the treatment of osseous fractures, defects, and ultimately diseases caused by, for example, bone tumor resection, accident trauma, and congenital malformation. Additive fabrication of stem cell-seeded, osteoconductive porous scaffolds has been an effective method in clinical practice for the treatment of bone pathologies (such as osteoporosis, osteoarthritis, and rheumatic diseases). Porosity is known to be one of the main morphological characteristics of bone tissues, which affects the functional performance of an implanted bone scaffold. Hence, in situ detection and quantification of scaffold porosity implemented to ensure functional integrity prior to implantation/surgery is an unavoidable need.

The presently-disclosed subject matter is thus based, at least in part, on the development of a robust, image-based method for the identification and subsequent characterization of the surface porosity and dimensional accuracy of additively manufactured bone tissue scaffolds, including those manufactured using a pneumatic micro-extrusion (PME) process. It was observed that the presented methods and systems are capable of detecting complex individual pores based on a micrograph. Moreover, using the proposed methods and systems, not only were scaffold pores detected, but also scaffold porosity was characterized on the basis of various defined quality metrics/traits (such as the relative standard deviation of distance to the nearest pore). The proposed method was further validated by contrasting its performance in “surface pore detection” against that of a standard method, tested on a complex benchmark in four different simulated lighting environments. Besides, the performance of the method in terms of “pore filling” was compared to that of a standard method, tested on a real PME-fabricated bone scaffold. It was observed that the proposed method had a better performance in pore filling, detection, and consolidation. In this regard, and without wishing to be bound by any particular theory or mechanism, it is believed that the presently-described systems and methods will advantageously further allow for the fabrication of patient-specific, structurally complex, and porous bone scaffolds with easily validatable, functional, and medical properties for the treatment of bone pathologies.

The presently-disclosed subject matter thus includes methods and systems for characterizing porosity of additively manufactured bone scaffolds. In particular, certain embodiments of the presently-disclosed subject matter relate to image-based methods and systems for characterizing pore size distribution and the dimensional properties of bone tissue scaffolds fabricated using additive manufacturing processes, such as pneumatic micro-extrusion (PME).

In some embodiments of the presently-disclosed subject matter, image-based methods and systems are provided for characterizing the pore size distribution and the dimensional properties of bone tissue scaffolds fabricated using the PME process. In some exemplary implementations, an exemplary method of the proposed invention is an image-based method that allows for the detection of scaffold pores and, in turn, a quantification of pore size distribution and an assessment of dimensional accuracy of bone tissue scaffolds. In some exemplary implementations of such methods and systems, a porous bone scaffold is first produced by an additive manufacturing process. Numerous additive manufacturing processes can be used in accordance with the presently-disclosed subject matter and are known to those skilled in the art, but will typically include a process of joining materials to make parts from three-dimensional model data and are usually performed in a layer-by-layer manufacturing arrangement. Such additive manufacturing techniques are inclusive of additive manufacturing process such as material extrusion, sheet lamination, binder jetting, material jetting, directed energy deposition, powder bed fusion, and Vat Photopolymerization, and can make use of solid, liquid, or powder-based material as a staring material.

In some particular implementations of the methods and systems of the presently-disclosed subject matter, the additive manufacturing process is a material extrusion process. In some implementations that make use of a material extrusion process, the materials extrusion process is a pneumatic microextrusion (PME) process whereby a high-pressure air flow is injected into the cartridge of a deposition head along with a polymer material in powder form. The material is then heated to a temperature above a melting temperature of the loaded polymer to provide a molten polymer that can subsequently be deposited on a substrate in a layer-by-layer fashion to produce a scaffold having a desired shape. In some implementations that make use of a material extrusion process, such as PME, to produce the scaffold, the material extruded is a biocompatible material or, in other words, a material that is substantially non-toxic in the in vivo environment of its intended use, and that is not substantially rejected by the subject's physiological system (i.e., is non-antigenic). In some implementations, the material is both biocompatible and biodegradable, such that the material is also capable of being broken down upon implantation. In some implementations, the material is a polyester-based polymer such as, in certain embodiments, polycaprolactone (PCL).

Regardless of the particular method and material used to produce the exemplary bone scaffold, once the bone tissue scaffold has been produced (i.e., subsequent to fabrication), one or more images of the scaffold are then captured and obtained. In some implementations, an image of an exemplary scaffold is obtained using a high-resolution charge-coupled device (CCD) camera such as, for example, a GS3-U3-91S6M-C CCD camera manufactured by FLIR Systems, Inc. Richmond, BC, Canada, as such a device allows high-resolutions images to be while making use of a lighting system capable of producing a sufficient lighting intensity and color temperature. However, it is of course contemplated that a number of different camera systems and other image capture devices capable of obtaining high-resolution images can also be utilized without departing from the spirit and scope of the presently-disclosed subject matter. In some implementations, to assist in the subsequent analysis of the image and as described in further detail below, the image captured from the high-resolution camera is a monochromatic image, such as a grayscale image.

Subsequent to obtaining the image of the produced bone tissue scaffold, to begin to analyze the image, the boundaries of the scaffold are then first determined to identify the dimensions of the scaffold. In some implementations, the scaffold bounds are determined by detecting an initial boundary with a bounding box. In drawing the initial bounding box, a focus in drawing the box is to bound the solid structure of the bone scaffold while discarding wisps of material, protruding strings, other artifacts of the additive manufacturing process. In this regard, in some implementations, bounded pixels are detected with each bounding box and then the bounding box is incrementally shrunken such that, during the shrinking process, an amount of material is discarded each time the bounding box is shrunk. To determine the appropriate boundary of the bone tissue scaffold in the image, a rate of change of discarded material is identified during the shrinking process and once that rate of change is equal to or exceeds a predetermined threshold parameter, such that a collision with a boundary of the scaffold or sample can then be assumed, the boundary of the scaffold is then identified or otherwise determined. In some implementations, this process for determining the boundary of the bone tissue scaffold is performed in an iterative manner whereby after an initial boundary is determined in one view of the image, the image is rotated, and the steps are repeated such that the boundary of the bone tissue scaffold with the least resulting area is identified and can be utilized for subsequent analysis. In some implementations, the bounding box is automatically drawn around the scaffold by making use of using a subroutine that initially draws a relatively large box, enclosing all visible material within the image, which is composed of black pixels (representing the surrounding) as well as white pixels (representing the scaffold). Subsequently, the box boundaries are incrementally shrunk, and the amount of material (i.e., white pixels) discarded in each incremental step is monitored. Once the amount of material discarded, referred to as the rate of change in pixel intensity from black to white, exceeds a specific threshold (e.g., a constant set by user), the right size of the bounding box is assumed, and the process terminates.

Upon identifying the boundary of the bone tissue scaffold in the image, the initial edges of any pores, highlights (i.e., reflections or the brightest or lightest elements in an image and which often correspond to raised or protruding features shown in an image), or other surface features are detected using an edge detection algorithm capable of detecting such surface features. Specifically, in some implementations of the edge detection process, a Canny edge detection algorithm is first utilized to generate an image in which the data can be represented as a set of discrete pixels that represent the edges, and in which those pixels are then each analyzed to obtain continuous edges that can be chained together until all edges are traced. Such a Canny edge detection algorithm can be performed in some implementations using commercially-available mathematical computing software such as MATLAB®, produced by The MathWorks, Inc. (Natick, MA). In short, however, and in such implementations, the Canny method finds edges in a 2D grayscale image by looking for local maxima of the gradient of the image, and then functions by calculating the image intensity gradient by taking the derivative of a Gaussian filter. In addition, in some implementations, the Canny method uses two thresholds (constant values) to detect strong as well as weak edges (when connected to strong edges) such that Canny method is: (i) less likely to be misled by noise, and (ii) more likely to detect true weak edges, when compared to the other methods. For additional information and guidance regarding the Canny edge detection algorithm, see, e.g., Canny, J., A Computational Approach To Edge Detection, IEEE Transactions on Pattern Analysis and Machine Intelligence, 8(6):679-698, 1986, which is incorporated herein by reference in its entirety. Of course, in some implementations, similar initial edge detection algorithms, such as Sobel, Prewitt, Roberts, and zerocross, can also be utilized without departing from the spirit and scope of the presently-disclosed subject matter.

In some implementations, as part of the detection of the various edges using an edge detection algorithm and because the edge detection algorithm is designed to identify sets of discrete pixels corresponding to an edge, an exemplary method of characterizing a bone scaffold in accordance with the presently-disclosed subject matter, can include a further step of identifying a continuous edge of the one or more detected surface features using a marching algorithm that makes use of a subroutine as described in further detail below. In this regard, and as shown for example in FIG. 20, pixel contacts are analyzed through a process by which pixels that are closest together in distance and whose direction is most similar to the continued direction are identified and the trajectory of such pixels is traced to thereby draw the continuous edge.

In some implementations, after tracing and upon the identification of the various edges in the bone scaffold shown in the obtained images, surface edges that correspond to highlights are filtered out to thereby identify the edges that correspond to the edges of a pore and so as to subsequently leave only, or a majority of, the pores to be computed and analyzed. In some implementations, to perform such a filtering step, a map of image intensities is calculated from the pixels in the image, and a gradient is calculated using the plurality of image intensities. In some implementations, this filtering out can then be, for example, accomplished by calculating gradients along a path and calculating any divergence along that path such that an angle difference in trajectory can be calculated and determined to be indicative of a highlight or pore. To further minimize any non-pore edges (e.g., straight line edges due to imperfections on the surfaces of the bone scaffold) from being passed through for further analysis, in some implementations, further processing of the detected edges is performed by checking the ratio of the width to height on those edges to thereby discard uncertain edges.

After the filtering step and to identify the pores, edges corresponding to a pore are identified from the mapping described above, and are subsequently compared to the corresponding pore in the original image and which were identified using the edge detection algorithm. Intersection of the pore edge from the mapping and the pore edge identified in the original image are then iteratively identified to thereby map and characterize the pore.

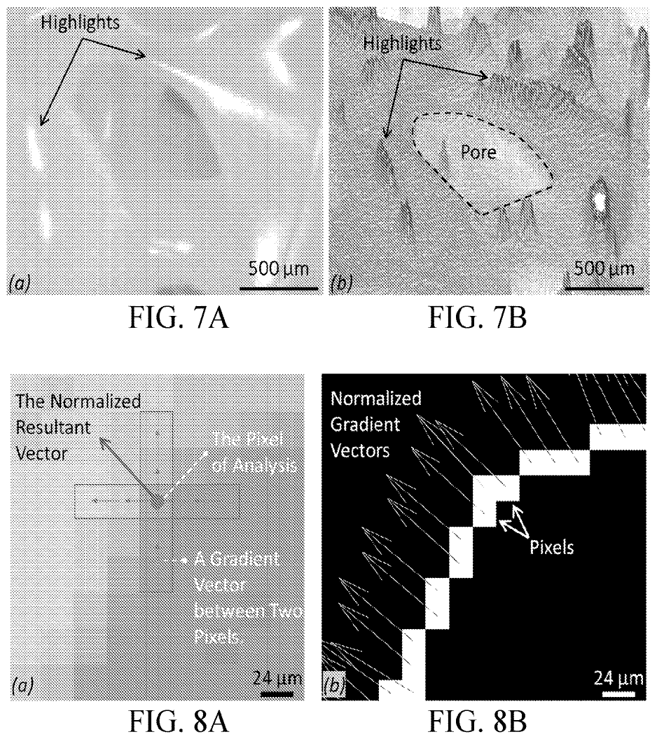

In particular, in some implementations, once the defining features of a pore are identified, a pore filling algorithm, which is a 3D height map technique, is utilized to identify the complex regions of a pore (having a gradual distribution of pixel intensity from dark pore regions toward bright surrounding scaffold regions). As illustrated in FIG. 7B, in some implementations, the pore regions are viewed as similar to a puddle such that the aim of the pore filling algorithm is to automatically fill the puddle until it spills over the edges of the pore. In particular, in the pore filling algorithm, the edges are first extracted into an image, while, in addition, the same region is extracted from the original grayscale picture (allowing for pixel intensity analysis). Subsequently, dark pixels (representing the pore region) are then extracted based on a threshold and analyzed to see whether the pixels align with the corresponding edges of the pore. Finally, the threshold is increased (with lighter and lighter pixels forming the “puddle”) until the intersection with the edges exceeds a predetermined value and then the process terminates. At this point in the process, the surface porosity can then be mapped and factors of surface quality can be determined, including but not limited to: the percent symmetry along the mid-x and mid-y axis after a 180° rotation; percent of surface consisting of porosity; average area of detected pores; and standard deviation of pore area. For additional information and guidance regarding the use of such a pore filling algorithm, which can also be referred to as a watershed technique, see, e.g., Beucher, S., “Use of Watersheds in Contour Detection,” Proc. Proceedings of the International Workshop on Image Processing: Real Time Edge and Motion Detection/Estimation, Rennes, France, Sep. 17-21, 1979, CCETT, which is also incorporated herein by reference in its entirety.

In some further embodiments of the presently-disclosed subject matter, a system for characterizing a bone tissue scaffold is provided that comprises an image capture device for capturing an image of the bone tissue scaffold; and a processor (e.g., a computer including a processor) for analyzing the image. The processor includes instructions for: determining a boundary of the bone tissue scaffold shown in the image; detecting one or more surface features of the bone tissue scaffold shown in the image using an edge detection algorithm, the one or more surface features including one or more pores and/or one or more reflections; identifying edges that correspond to a pore shown in the image using a pore filling algorithm; and mapping the surface porosity of the bone tissue scaffold shown in the image.

With regard to the processor included in the systems of the presently-disclosed subject matter, it is of course appreciated that the processor is programmed, in certain implementations, to execute instructions stored in a memory component or other computer-readable medium to process the images acquired by the image capture device (e.g., camera) and for carrying out the steps of the methods described herein. Of course, it is also contemplated that the processor described for use herein can also comprise multiple processors with particular processors capable of carrying out various steps in the described methodologies.

By making use of the above-described methods and associated algorithms, the presently-disclosed methods and systems are thus capable of providing layer-by-layer analysis of scaffolds and other products produced by additive manufacturing. Moreover, in some embodiments, the presently-disclosed systems and methods can be included in firmware for three-dimensional (3D) printers to allow for process monitoring, and can be extended to analyze pore size distribution and the dimensional properties in other applications and materials as well, including dental structures and applications, construction applications (e.g., analysis of metal structures), and the like.

The presently-disclosed subject matter is further illustrated by the following specific but non-limiting examples.

### EXAMPLES

The porosity of PME-fabricated bone scaffolds, captured by a micrograph, is intrinsically composed of a wide range of complex gradual pores characterized with intensity gradients and no discrete, sharp edges. In fact, it is not easy to identify the regions near an edge and thus to define regions corresponding to a pore from a single micrograph. Hence, “topographical or depth mapping” (proposed in this work) is an important step toward identification of gradual pores and quantification of scaffold surface porosity. In the absence of such a topographical mapping-driven method, the detected pores of a bone scaffold may significantly overlap or be internally discontinuous/heterogeneous, resulting in inaccurate quantification of scaffold porosity (in terms of, for example, estimation of surface area per volume, pore size distribution, and material diffusion rate) as well as subsequent cell-scaffold interactions.

Topographical maps of images with intensity (represented as altitude) will be presented in this work as a component of the porosity filling algorithm. This concept is used for analyzing the porosity of complex PME-fabricated scaffolds, as a principle for identifying porosity in a wide range of lighting conditions, and on a wide range of pore geometries (most specifically on pores, which are gradually forming without a discretely defined boundary).

Another principle of this work, which sets it as an improvement above the standard methodologies, is the robustness of the algorithm to different, complex, real-world lighting conditions without calibration. An image processing technique, which is often used to solve this problem, is Otsu's method from a 1979 work. This was used by Bowoto et al.; however, it only works when there are clearly defined dark and light regions with a clear partition in intensities. For complex, gradually forming indentations in variable lighting conditions, there are often more than discrete groupings of intensities, which cause Otsu's method to fail.

Another problem addressed in this work is the challenge of variable lighting conditions across a single image, to where a single threshold cannot be set. The common image processing technique, used to solve this, is known as “adaptive thresholding”. The problem with using adaptive thresholding, however, is that it requires a predefined ratio of dark to light, and if that is not available, then it requires the use of Otsu's method with the aforementioned challenges. See, e.g., Otsu, N., 1979, “A Threshold Selection Method from Gray-Level Histograms,” IEEE Transactions on Systems, Man, and Cybernetics, 9(1), pp. 62-66, DOI: 10.1109/TSMC.1979.4310076, which is also incorporated herein by reference. All in all, the proposed work uses a new technique of (i) analyzing porosity on a pore-by-pore basis, (ii) filling pores (until overflow is detected) using the watershed technique, and (iii) analyzing the properties of the fill.

With the above in mind, a goal of the research was to fabricate biocompatible, biodegradable, and porous bone tissue scaffolds, designed to incorporate autologous human bone marrow mesenchymal stem cells, for the treatment of osseous fractures, defects, and structural deformities in clinical practice. In pursuit of this goal, experiments were thus undertaken to produce a robust, image-based method for the identification and subsequent characterization of the surface porosity and the dimensional accuracy of additively manufactured bone tissue scaffolds, with a focus on pneumatic micro-extrusion (PME) process. The aim is (i) to detect complex surface pores and (ii) to characterize scaffold surface porosity using a range of developed quality metrics/traits based on top-layer or layer-by-layer micrographs acquired from PME-fabricated scaffolds.

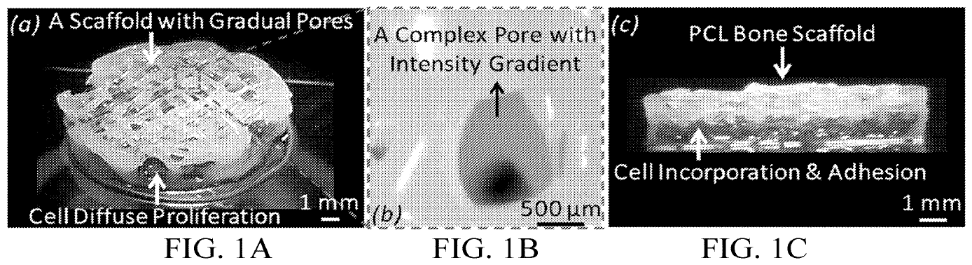

While several image processing techniques have been presented in the literature, there are few works, which may be capable of detecting and characterizing the complex morphology and the intrinsic gradual pores (defined as pores that are not discretely, completely bound) of PME-fabricated bone scaffolds as exemplified in FIG. 1; note that due to the unique depth of the scaffold pores, there is an intensity gradient associated with each pore, leading to formation of edges that are not clearly defined. The method implemented in this work is unique in that it is the only method known, which can detect and label gradually forming pores from a single camera image under a robust set of lighting conditions. In addition, a set of morphology/porosity metrics are introduced and integrated into the algorithm for quantitative characterization and analysis of scaffold surface porosity; it is shown that the porosity metrics also have correlation with scaffold mechanical (and other functional) properties.

The proposed method allows for a comparison between the porosity of a layer-by-layer fabricated scaffold and that of a reference model and thus verifying whether the newly fabricated scaffold meets porosity/morphology requirements or not. Such a capability, in addition, will pave the way for near real-time characterization of scaffold dimensional accuracy as well as process monitoring by determining (i) how well a printed scaffold matches its target CAD geometry and (ii) how deviation from the target geometry is likely to weaken the fabrication process (given the same material deposition rate).

### Material and Methods

Pneumatic Micro-Extrusion (PME) Process. PME is a high-resolution material-extrusion additive manufacturing method, which has been widely utilized for the fabrication of biological tissues, structures, and constructs. Having a layer resolution of 100 m, the PME process allows for non-contact, multi-material deposition of a wide range of functional bio-inks for tissue engineering applications. However, the PME process is inherently complex (highly nonlinear), governed by complex multi-physics phenomena (e.g., phase-change, coalescence, receding-relaxation, and wetting equilibrium). The PME complexity, in addition, stems from the presence of a broad spectrum of process parameters—e.g., material viscosity, flow pressure, material deposition temperature, and solidification rate—as well as material-process interactions. Consequently, investigation of the effects of significant process parameters (and their interactions) in addition to assessment of the dimensional and functional properties of fabricated bone scaffolds would be required for optimal fabrication of mechanically strong, dimensionally accurate, and patient-specific bone scaffolds.

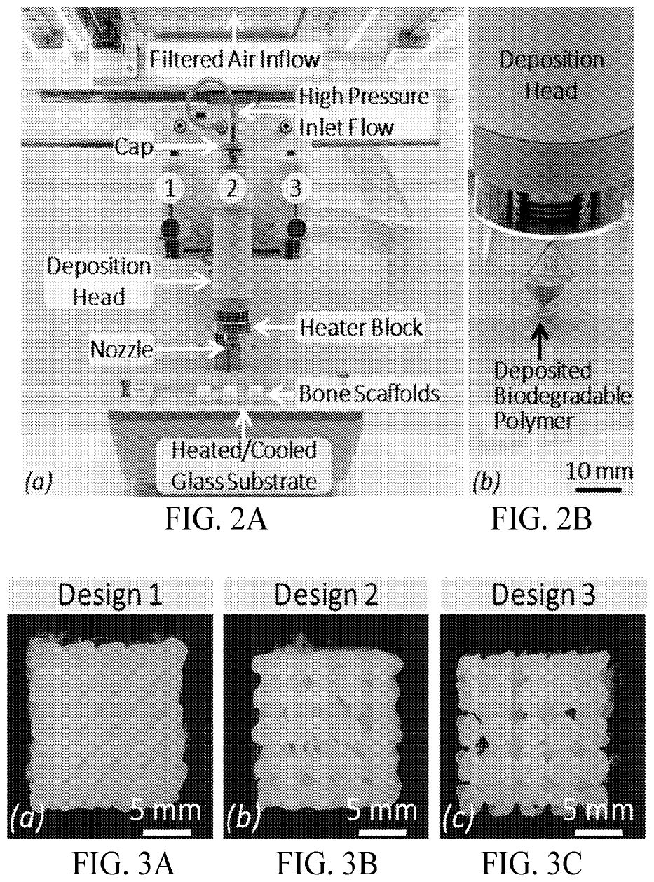

The medium of transport and deposition in the PME process is a high-pressure flow of air, supplied by a compressor. As demonstrated in FIG. 2, the high-pressure inlet flow is injected into the deposition head's cartridge, loaded with a polymer material in powder form and heated to a temperature above the melting temperature of the loaded polymer; this leads to formation of a molten polymer flow in the cartridge prior to deposition. Subsequently, the molten polymer flow is deposited on a heated/cooled substrate via a converging micro-capillary nozzle. With the aid of a computational fluid dynamics (CFD) model, others have demonstrated that the transport of molten PCL through a micro-capillary nozzle (200 μm in diameter) under a flow pressure of 550 kPa would be a viscous flow, characterized with a Reynolds number (Re) of <<1. This is unlike other direct-write additive manufacturing methods (e.g., aerosol jet printing) where material deposition is intrinsically turbulent. Filtered air is delivered to the chamber at a constant rate using a fan, which aids in maintaining a fixed level (rate) of polymer solidification (and thus layer adhesion) after deposition. In general, an optimal substrate temperature is critical for proper layer adhesion and thus accurate material deposition.

Material. Composed of PCL (Cellink, Boston, MA, USA), porous bone scaffolds were fabricated using the PME process. A polyester-based polymer, PCL is derived from caprolactone monomer using ring opening polymerization. It is not only biocompatible and biodegradable, but also semi-crystalline and hydrophobic. As discussed below, an aspect of this work is to treat osseous fractures. Hence, fabricated bone scaffolds need to be biocompatible (to create a non-cytotoxic environment for cell growth and diffuse proliferation) as well as biodegradable (to facilitate scaffold-free, autologous bone remodeling and metabolism). It has been observed that scaffold hydrophobicity, in addition, is a significant design factor that enhances the treatment of bone defects.

In this study, the PCL material (in powder form) was used as received. It has a molecular weight (Mn) and density of approximately 80,000 and 1.145 g/mL (at 25° C.), respectively. In addition, PCL has a melting temperature in the range of 59-64° C. as well as a glass transition temperature of −60° C. Furthermore, PCL has a tensile strength and elasticity modulus of 16 MPa and 0.4 GPa, respectively. The PCL material was stored in a freezer at −80° C. prior to consumption to prolong the shelf life of the material (approximately one year).

In the second portion of experiments, bone scaffolds were instead fabricated using a hydroxyapatite-polysaccharide starch material. This was to allow for more rapid material deposition and data collection as the second portion of experiments, consisting of layer-by-layer image acquisition. The material was synthesized with 11.42% polysaccharide potato starch (Pure Supplements Co., Lindon, UT, USA), 8.43% hydroxyapatite (CAS number: 1306-06-5, molecular weight: 502.31, Sigma-Aldrich, St. Louis, MO, USA), and 4.00% titanium dioxide by mass (merely used to change the color of the synthesized materials from translucent to opaque), with the remainder of the weight consisting of water.

Experimental Design. As demonstrated in Table 1, a single-factor experiment was designed and conducted with the aim to fabricate bone scaffolds with three levels of porosity, i.e., low, medium, and high (in order to assess the performance of the proposed pore detection method). The fabricated scaffolds were correspondingly based on three triply periodic minimal surface (TPMS) designs, i.e., Schwarz Diamond (Design 1), Schwarz Primitive (Design 2), and Neovius (Design 3). Note that TPMS surfaces repeat periodically in 3D and intrinsically have zero mean curvature; in other words, at each point, the sum of the principal curvatures is zero. Others have investigated the mechanical properties of a wide range of TPMS designs. Being of two intertwined congruent labyrinths, the Primitive design resembles an inflated tubular cubic lattice, while the Diamond design resembles the diamond bond structure (i.e., tubular and inflated in shape) consisting of two intertwined congruent labyrinths. Both designs have high surface-to-volume ratio and thus are suited for the fabrication of porous scaffolds. In addition, the Neovius design (as a cubical unit cell) is composed of outward necks that are extended toward the middle of each edge. The Schwarz Primitive design, the Schwarz Diamond design, and the Neovius design are mathematically defined by Equations (1)-(3) respectively, as follows.

$$cos x + cos z + cos y = 0 \qquad ( 1 )$$

$$sin x sin y sin z + sin x cos y cos z + cos x sin y cos z + cos x cos y sin z = 0 \qquad ( 2 )$$

$$3 ( cos x + cos y + cos z ) + 4 ( cos x cos y cos z ) = 0 \qquad ( 3 )$$

Listed in Table 1, the PME process parameters were set at their optimal values, based on the authors' prior characterization studies. PCL powder was loaded into the cartridge, maintained at 180° C. To ensure steady-state and uniform material deposition, the loaded PCL was kept in the heated cartridge for 15 minutes prior to deposition. Supplied by an oil-less, rust-free air compressor, the flow pressure was set to 300 kPa. Laminar molten PCL flow was deposited on a cooled glass surface (uniformly kept at 10° C.) with a print speed of 2.5 mm/s, using a 200 m nozzle. The scaffolds were fabricated using a layer height of 200 m, as illustrated in FIG. 3.

TABLE 1
PME process parameters, defined in this study, for
the fabrication of porous bone tissue scaffolds.

| | | |
|---|---|---|
| Parameter | Type | Level [Unit] |

Variables

| | | |
|---|---|---|
| Scaffold Design | Design | 1. Schwarz - Diamond (Design 1) |
| | | 2. Schwarz - Primitive (Design 2) |
| | | 3. Neovius (Design 3) |

Fixed Parameters

| | | |
|---|---|---|
| Scaffold Dimensions | Design | 15 × 15 × 15 [mm] (Cubic) |
| Layer Height (Thickness) | Design | 200 [μm] |
| Infill Pattern | Design | Concentric |
| Nozzle Size | Machine | 200 [μm] |
| Bed Temperature | Machine | 10 [° C.] |
| Print Speed | Machine | 2.5 [mm/s] |
| Deposition Head | Machine | 180 [° C.] |
| Temperature | | |
| Deposition Flow Pressure | Machine | 300 [kPa] |
| PreFlow Delay | Machine | 900 [ms] |
| PostFlow Delay | Machine | 400 [ms] |

The geometry of the fabricated scaffolds is cubic, having dimensions of 15×15×15 mm. The PME platform used in this study was BIO X (Cellink, Boston, MA, USA). In addition, DNA Studio (Cellink, Boston, MA, USA) was the slicer software of choice, used to convert the 3D models into a G-code and thus to create a tool-path for fabrication. Exemplified in FIG. 3, monochromatic images were captured off-line from each scaffold using a high-resolution (9.1 MIP) charge-coupled device (CCD) camera (GS3-U3-91S6M-C, FLIR Systems, Inc., Richmond, BC, Canada). The imaging system is illuminated by a LED ring light having a brightness of 30,000-40,000 Lux and a color temperature of 6000 K (AmScope, Irvine, CA, USA). The acquired images were saved as raw8 data format and .tif file format. In the second part of experiments, the layer-by-layer images were captured in situ (after material deposition) using the process camera toolhead (Cellink, Boston, MA, USA) mounted on the PME system with the chamber lights on.

The compressive properties of the fabricated bone scaffolds were measured using a compression testing machine (MTI-10K, Measurements Technology Inc., Marietta, GA, USA). As shown in FIG. 3, the fabricated scaffolds were cubic, having a gauge length of 10.5 mm. For the compression test, they were placed between two compression plates, subjected to a compression load applied with a rate of 0.08 mm/s. The elasticity modulus of each specimen was accurately calculated using a computer program developed in the MATLAB environment, processing the stress-strain data points obtained from the compression test.

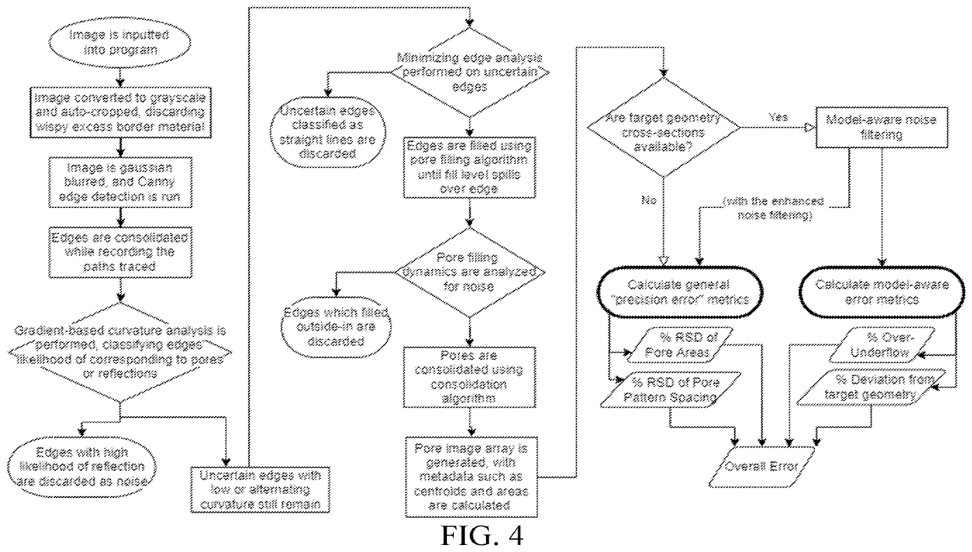

Image-Based Scaffold Pore Characterization. The following will describe methods to find the dimensional boundaries of a 3D printed bone scaffolding sample; subsequently, the process of edge detection, continuous edge identification, pore-highlight discrimination, and then the methods of filling identified pores. Finally, there will be a discussion of the final porosity analysis and validation. FIG. 4 illustrates a flowchart, detailing the main steps of the proposed method for the characterization of porosity and analysis of dimensional accuracy of additively manufactured bone tissue scaffolds.

Scaffold Bounding. The aim of scaffold bounding is to find the initial dimensions of a scaffold. The photos of the samples are slightly rotated in one direction or the other, and the sample photos contain noise in the form of material threads leftover from the PME process. Therefore, a simple method was devised to effectively bound the samples for the purpose of dimensional measurements.

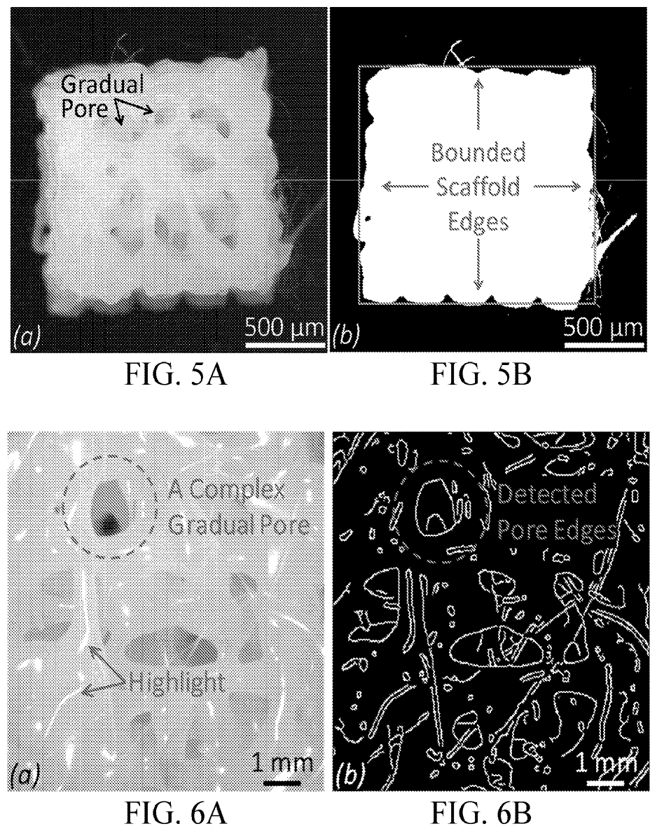

Initially assuming the image is correctly rotated, the objective is to find the correct positioning for a bounding box, bounding only the solid scaffold structure, discarding the slight wisps of material and protruding strings, as seen in FIG. 5.

To start, an initial bounding box encloses all visible material within the image, but in order to discard noise, the box boundaries are incrementally shrunk, monitoring the amount of material discarded in each incremental step. Once the rate of change of bounded material within the bounding box exceeds a specific threshold parameter, collision with the solid structure of the sample is assumed, and the process terminates.

To find the optimal image rotation, the bounding process is performed multiple times at different angles, and through successive approximations, the angle which results in the bounding box with the least resulting area is used, as the structure is now correctly rotated.

Edge Tracing and Consolidation. The initial processing step to each bone scaffolding printed sample is to utilize MATLAB's Canny edge detection algorithm to find the initial edges of what could be pores, highlights (reflection), or other surface features, as illustrated in FIG. 6. The Canny edge detection has a thresholding parameter which defines how sensitive it is in defining edges along differences in pixel intensities. A threshold is manually selected to be the minimum needed to identify all major surface pores; as going beyond the minimum, the threshold introduces unnecessary noise from surface highlights. This step needs only to be done once given specific lighting conditions and is generally inconsequential to final measurements, as the following algorithm is quite robust to discarding edges not corresponding to surface pores. Before the Canny edge detection algorithm was run, a slight Gaussian blur was added to assist in reducing noise in the scaffold image. The usage of the Canny edge algorithm is as follows: first edges are grouped into continuous edges, the curvature of these edges is analyzed in comparison to image intensity, and then, the curvature is used to determine pores, highlights, and those features which need further analysis.

Once an image is generated through Canny edge detection, the data representation is merely a set of discrete pixels representing edges of contrast. To begin, because a continuous edge often corresponds to a unique surface feature, a marching algorithm is used to chain continuous edges together by analyzing pixel-by-pixel contacts, resulting in a list of separate non-contacting edges. The marching algorithm is more sophisticated than needed for simply grouping contacting lines of pixels. As the algorithm traces the edge, the path trajectory is monitored in order to simultaneously record the curvature along the current point of the edge, which will become relevant in the next step. Details on the path trajectory and the curvature calculation are delineated below.

Scoring Viable Next Pixels While Following an Edge. In order to select the next pixel in the chain, all possible pixels must be scored in case there are multiple options available, as shown in FIG. 20; the objective is to favor pixels, which are closest in distance and whose direction is most similar to the present average direction. First, each viable pixel's distance from the current pixel is calculated, with directly adjacent pixels having a distance of 1 and diagonal pixels having a distance of approximately 1.41. Subsequently, to calculate similarity of trajectory, the running average of the direction vectors of the previous few pixels in the chain is calculated, with the direction vectors being shown in FIG. 20. The average direction is then compared to all the new possible directions through the use of the dot product. Finally, the score of each pixel is calculated by taking the negative of the distance and adding it to one-fourth of the dot product, with 14 being the factor in order to reduce the impact of direction so that minimal distance is always favored.

Technically, if no scoring were performed, the edge labeling algorithm would still be fully capable of finding all connected edges. However, the purpose behind such scoring will become clear in the following discussion of edge curvature analysis. The average of the previous 8 pixels direction differences is used to calculate direction. Its selection was roughly through trial and error according to the following criteria: while current direction should be as tangential as possible to the current path, due to the pixelized discrete nature, a small pixel history is used to smoothen out the edges. Small particles on the surface are blurred and less likely to be detected, while larger features (such as pores) become smoother, resulting in more continuous edge detection.

Arbitrary Starting Points and Handling Skipped Portions of Edges. Up until this point, the method described has specified what takes place given a starting pixel, and nothing has been mentioned as to what happens to a skipped off-shoot of an edge due to a branching point, as schematically shown in FIG. 20. At the beginning of the process, after edge detection, it was mentioned that x-y coordinates of every pixel from the image generated by the Canny-algorithm were placed in a list. Simply, the first pixel used to begin the edge tracing is the first pixel from that list, and then after that, edge trace terminates due to a dead end; the next starting pixel selected is the next unlabeled pixel in that list. The result is that no matter what edge is skipped due to a branching point or due to starting in the middle of an edge, the starting point of the next pixel is located somewhere on the unlabeled edge; hence, inevitably all edges are traced, regardless of the arbitrary starting pixels.

Initial Pore-Highlight Discrimination through Gradient-Based Curvature Analysis. Early on, it is important to filter out as many surface edges as possible which correspond to highlights, as opposed to pores to minimize computation time and to minimize the noise passed through to the next stages of the algorithm, which will perform further analysis.

From an initial consideration, if the curvature along a continuous path and divergence of the image intensity could be calculated, then it would be simple to discriminate between pores and highlight surface features. This could be done by stepping along the path, taking the dot product of a curvature vector (pointing inwards along the radius), multiplied by the gradient vector, followed by integrating the result. However, due to the non-continuous nature of the problem, with the intensity being represented by individual pixels on a grid, there is more nuance in the process. In FIG. 7, a 3D height mapping of image intensities is shown for further reference on how the intensity gradient corresponds to pores as indentations and highlights as tall peaks.

The gradient of image intensity must first be calculated along every point of the edge being analyzed. Due to the discrete nature of a raster-pixelized image, calculating the gradient is done as an approximation. The aim is to calculate the general direction of intensity difference. It is calculated by taking the average gradient along the x-axis, two pixels ahead and two pixels behind the target pixel; this gives the x-component of the gradient at that pixel. In addition, the same is done to calculate the y-component of the gradient. A visual diagram is given in FIG. 8(a) of the method described, and the resulting gradients are shown in FIG. 8(b). Note that in both parts of the figure, the gradient vectors are normalized to more easily visualize the direction of the gradient.

Using the calculated gradients, and calculated curvatures of the path along each edge, a numerical value is calculated. Due to the discrete nature of raster images, and the specific objective of merely discriminating between pores and highlights, divergence along the curve is calculated in a modified manner for this problem. To begin, the algorithm starts marching through individual image pixels along the detected edges. As the algorithm marches along the edges, pixel by pixel, of each surface feature, referring to FIG. 20, the difference in the angle between the average direction vector and the newly selected direction is calculated at each step along an edge. The sign convention used is determined as follows: a delta difference vector is calculated between the new direction selected and the average direction vector; the result is dot-product multiplied with the intensity gradient vector at that point along the edge, and if the result is positive then the angle is positive, if the result is negative the angle is negative. Thus, macroscopically, the angle difference in trajectory is positive if the trajectory is curving towards the gradient, as in a highlight.

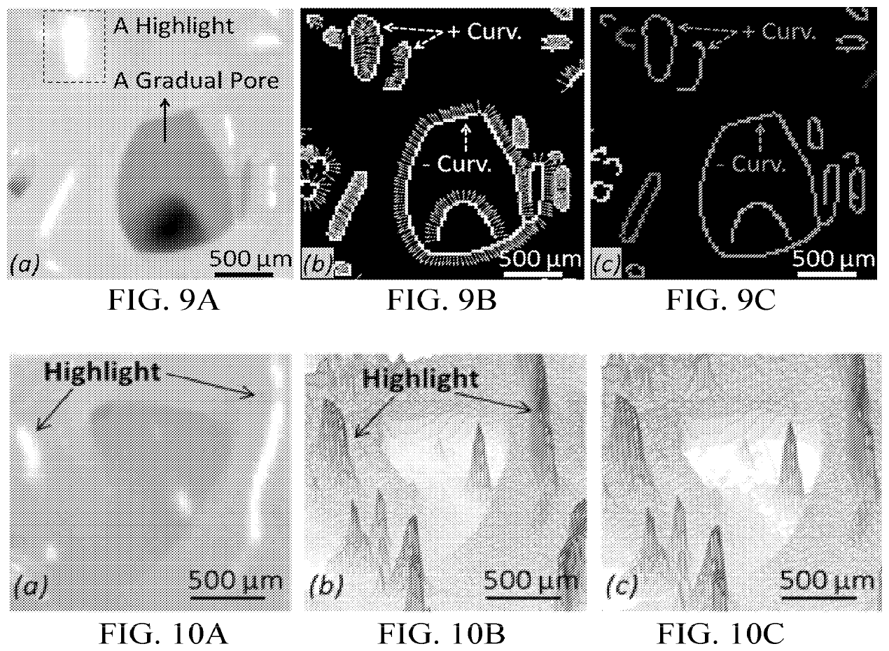

By taking the average trajectory angle difference along each point of an edge, with the sign convention described above, a discretized version of divergence can be calculated along an edge, and because the image intensity gradient affects the sign convention only, it would be more accurate to refer to the calculation as curvature towards the intensity gradient from this point forwards. The result is shown in FIG. 9.

At this point, while most edges have been classified as a pore or a highlight, some edges have values with insignificant leaning to either end of the range of values. These are usually in the form of straight line edges due to a surface imperfection on the bone scaffold. To filter these out, a simple post processing step is done which checks the ratio of width to height of unclassified features, easily discarding straight edges before continuing. Details on discarding uncertain edges via analysis of minimizing dimensions are given below.

Discarding Uncertain Edges via Analysis of Minimizing Dimensions. Some edges have uncertain curvature averaging near zero due to some pores having ‘S’ shaped edges, where for some of the edge, the curvature is bounding a pore, while for the other portion of the edge, the curvature is bounding a highlight or surface protrusion. Also, it is hard to discriminate between straight lines with little curvature and edges with varying curvature along the path. To solve this problem in the next stage of the algorithm, in order to minimize the amount of non-pore edges passed on to the next step, the minimal bounding dimensions of the edge are considered.

An edge with little curvature, a straight line, would have a minimal bounding box with a much more extreme ratio of width to height. As in, a straight line may be minimally bounded by a box with a dimensional ratio of 7:1, while an ‘S’ shaped edge with the same curvature could only be bounded by a box with the ratio of 3:1. The challenge is that the edges are not oriented in such a way that a bounding box would be the minimal bounding box, therefore, before all edges can continue on, any edge with a curvature of ±a threshold are further inspected.

The method of inspection is to extract the suspect edge into an image, and simply through a method of successive approximations; the edge is rotated and bounded, repeatedly, in the direction of greatest decrease in the area of the bounding box, until a minimum acceptable tolerance is achieved. This method is highly effective in discriminating straight lines as compared to ‘S’ shaped edges. For the algorithm, the minimum acceptable ratio used is 1:4, found by observing the ‘S’ shaped edge with smallest ratio, which was approximately 0.27. Note that the first few steps in the marching algorithm along an edge are weighted very little in calculating the curvature, as the average trajectory along those points is still highly variable, due to the discrete nature of the image and method of calculating trajectory.

Pore Filling Algorithm by Thresholding Until Collision. The purpose of this step was to analyze the surface porosity and quality of a fabricated bone scaffold; note that the edge-analysis described above is simply a precursor step. It can be observed from FIG. 9 that the defining feature of a pore is of darker appearance than that of its direct surroundings. However, thresholding the image, selecting pixels below a specific brightness, and calling them a pore are not sufficient, as different regions of the image require different threshold limits and it is not trivial to define such. Therefore, the first purpose of defining unique edges corresponding to pores was to be able to go through on a pore-by-pore basis.

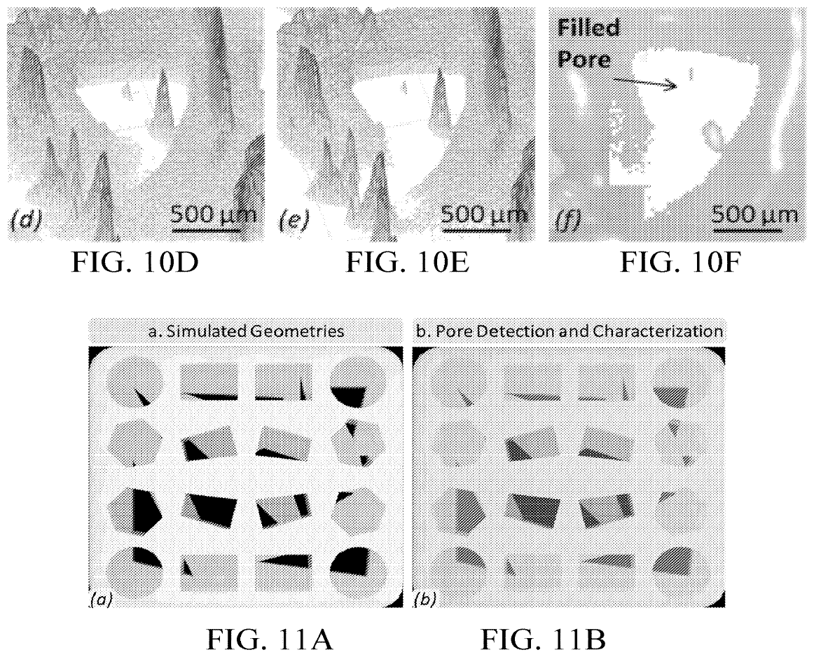

The major challenge of this work was that there was not a simple way to define regions, which correspond to a pore and which do not, as the majority of the pores are gradual, as in, they are not completely bounded; instead, they tend more to “spill in” being bounded by a ‘C’ shaped boundary, as shown in FIG. 7. As a result, it can be observed in FIG. 10, in the 3D height map, that it is similar to an indentation in a surface, and if it were to be filled with water, similar to a puddle, it could be filled until it spilled over the edge. Therefore, that was the direct approach taken in the following steps described below, in order to identify the regions near an edge that correspond to a pore. For further information relating to 3D height map techniques, referred to as the “watershed technique”.

Given all edges which have the possibility to bound pores, the pore filling algorithm functions as follows: (i) The pixels drawing the edge is extracted into an image and the same region is extracted from the original grayscale picture of the printed bone scaffolding sample. (ii) Darker pixels below an initial threshold from the greyscale image are extracted. That thresholded pore is checked for “complete intersection” with the corresponding edge. (iii) The threshold is iteratively increased, with lighter and lighter pixels forming the “puddle”, until the intersection with the edge exceeds a certain amount, and the process terminates, when the filling has spilled over.

Note that for strangely shaped pores, a robust intersection algorithm was developed named “complete intersection”, as detailed below. In addition, there is a final major noise filtering step (following the pore filling), which checks whether pores are filled outside-in (instead of inside-out, in which they are labeled as noise and omitted). The noise filtering step also has been delineated below.

Method of Calculating Complete Intersection. Initially in the development of this algorithm, the method of calculating intersection between the filling (the thresholded image) and the edge was simply to check direct overlap between them, however, because a single parameter controls the percent intersection limit on all pores, the challenge was that some edges suffered substantially far more intersection early in the filling process due to protruding edges which spiraled into the region which should be filled. This is usually caused by pores within pores, as the Canny edge-detection algorithm tends to connect them into a single edge.

A method was developed to treat edges with and without internally branching portions the same, by requiring that a ‘complete intersection’ occur before being included. Essentially, instead of counting the number of edge-pixels which are overlapped by the filling, the edge-image is broken up into rows and columns, where instead, the algorithm counts the total number of rows and columns experiencing all of their corresponding pixels in intersection.

For example, in a rectangular edge, there would be 2 pixels contained in each row and 2 pixels in each column, except at the perimeters. If the rectangle has the dimensions of 20 by 30 pixels, then there would be 50 rows and columns with the potential of intersection. The only way for any row or column to be counted as completely intersected is if all the pixels within them are overlapped. Therefore, with the actual overflow parameter used being around 50%, the algorithm will continue to increase the filling threshold until 50% of the rows and columns of the rectangular edge experience a complete intersection, requiring both parallel sides of the rectangle to be intersecting. The outcome of this method of determining complete intersection is that any stray edges spiraling into the rectangle example above have no impact on the outcome, as the intersection must take place end to end along a row or column, regardless of an internal edge.

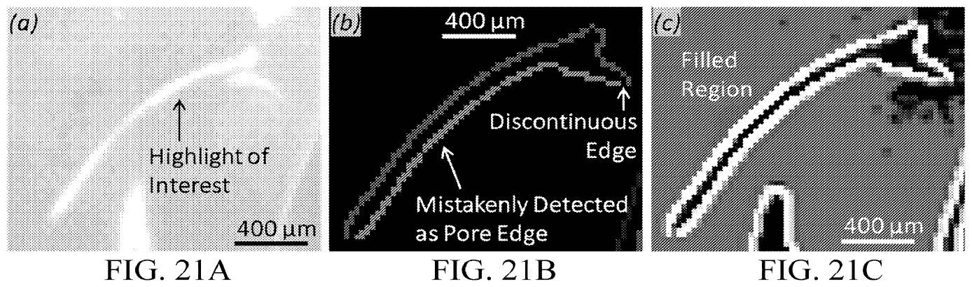

Final Discrimination of Edges by Analysis of Fill. Lastly, before the porosity fillings are all compiled and passed on for analysis, there is a final quality control test to ensure that no edges corresponding to straight edge surface features nor highlights can contaminate the data. Shown in FIG. 21 is an example of a pair of two edges which were labeled with no significant curvature due to a break (discontinuity) in the continuous Canny-detected edge right before the U-turn which would have skewed its curvature calculation to that of a highlight. In addition, the substantial bend resulted in it passing the minimal dimension ratio test. Fortunately, there is an obvious feature of highlights, when imagining the filling process on the 3D intensity heightmap of FIG. 10, which is that they fill outside in.

A final analysis is performed after the filling process on all fillings, if the percent collision with the outer boundary of the region corresponding to the edge exceeds a specific parameter (around 55%), the filling is flagged and discarded. This method of only accepting pores is the most effective barrier to preventing noise from affecting the final analysis, in fact, it is effective enough to accomplish most of the task of discarding highlights and straight edges on its own, with the previous checks only assisting to reduce computation time and to reduce noise passed on. Note that the fillings which intersect with the image perimeter are discarded, for they are most often just a result of indentations along the perimeter of the top surface of the pore, as there are no pores which could be on the perimeter of the sample.

Analysis Methods of Produced Surface Porosity Mapping. At the end of the process, a mapping is produced with color-coded, distinct pores in an array of images which are compiled together and consolidated; note that if two fillings overlap, they are consolidated. Using this image array, many metrics of surface quality can be determined. There were two routes of image analysis taken in the pursuit of quantifying quality using this array of pore images. One route taken of quantifying quality consists of calculating consistency metrics of the pore image array such as variation in pore areas and pore distances, while the other, more comprehensive route, compares the produced pore image array directly to the bone scaffold model geometry.

Given data on each individual pore on the surface of the bone scaffold as shaded regions of a grid, the quality of bone scaffold can be quantified to a significant extent without even referring to the target geometry of the surface. The reference-independent analysis to be described, makes the assumptions that the surface pores are regularly spaced at a consistent pattern, and that all pores on the surface of the scaffold are expected to be at the same size.

Thus, two metrics are produced from the reference-independent analysis step: the relative standard deviation of pore areas (rsd_area), and the relative standard deviation of nearest-neighbor distance (rsd_distance). More specifically for the latter, every pore on the surface is iterated over and the distance to the closest pore is added to a list. The relative standard deviation is then calculated over these distances. The weighted average of both the “rsd_area” and “rsd_distance” metric is then taken and referred to as the “precision error” in the rest of this work. The ratio is 1:2 respectively. A perfect printed scaffold would have zero standard deviation in both metrics, assuming the model geometry met the original assumption stated above.

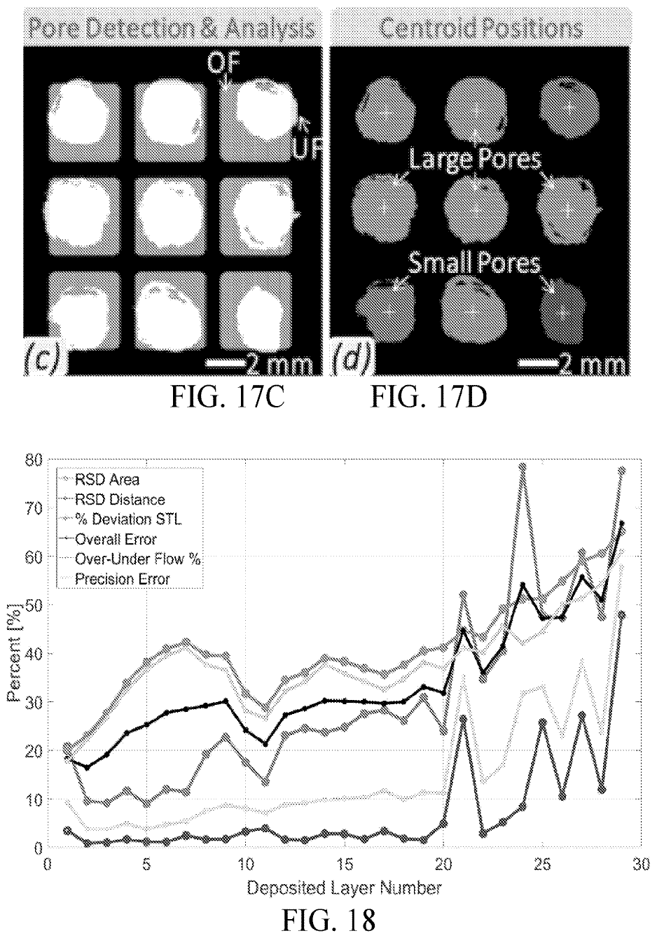

The second route of analysis involves comparison to the target model geometry. This is achieved by taking a top-down projection of the target model geometry to extract a shaded cross-section. The cross-section image is then aligned with the actual surface pore shaded image data, and the overlap can then be studied. To ensure that the two images are properly aligned, their alignment is iteratively optimized until an alignment is found which minimizes the area not overlapping between the two images (as will be illustrated later in FIG. 17.

The advantage of this reference-dependent analysis route is that it can determine between insufficient material deposition or excess material deposition with a quantitative amount. If the printed pores are too large compared to the target geometry, meaning that insufficient material was deposited, then the data is representing under-extrusion, or if the printed pores are too small compared to the target geometry, representing over-extrusion. This extrusion error can then be quantified by a single metric, called “percent over/under-extrusion”, with over-extrusion being positive, and under-extrusion being negative. An additional metric is calculated, called “percent deviation from reference”, which takes the absolute value of all deviation from the target model. The outcome of this is that while a model with half the pores over-extruded, and half the pores under-extruded may net zero overall error, the “percent deviation from reference” metric would quantify the absolute error.

### Results And Discussion

Validation of the Method. During development of the work, in order to validate the method developed, a test sample with known parameters was designed and 3D rendered with a similar opaque white material with protrusions and pores behind the pores, as illustrated in FIG. 11. The sample was designed with a surface porosity of 38.22%, and a dimensional ratio (width to height) of 1:1.1. The proposed method detected and labeled every unique pore on the surface of the simulated sample (shown in FIG. 11). In addition, it calculated a surface porosity of 38.12. The measured dimensional ratio through the initial sample bounding was 1:1.099 (width to height).

However, this simple model was not complex enough to validate the method proposed in this work. One of the claims of this work is that in regards to quantifying the quality of a printed scaffold with a single photo, the method proposed is claimed to be robust to different lighting environments and materials, functioning with a single set of parameters, and solving for the ones which remain. In order to demonstrate this, a more thorough validation was then performed.

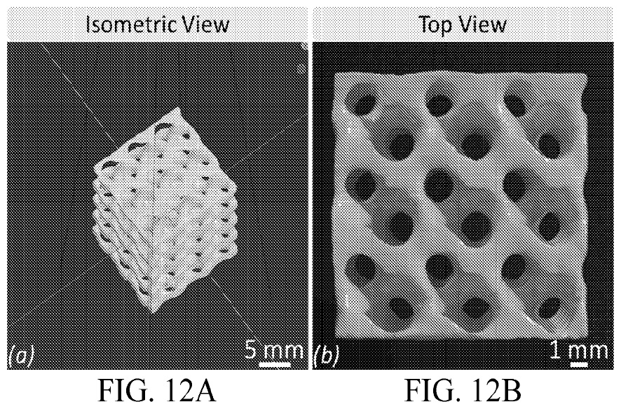

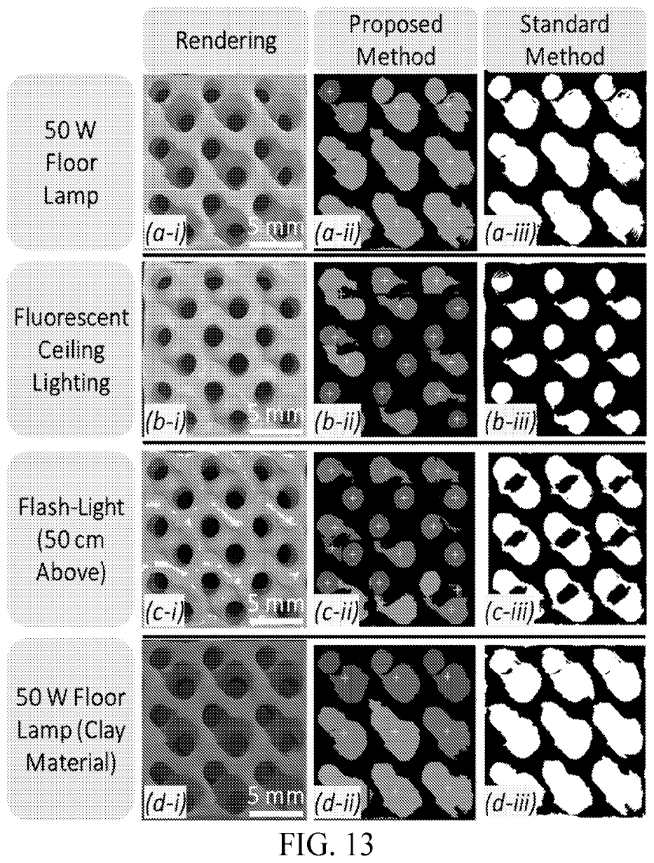

In FIG. 11, the pores are clearly defined, and the model has no complex noise and geometry beyond the few edges placed behind the pores. In addition, this model does not contain any pores which gradually develop and are vaguely defined. As a result, a more robust approach was taken by directly implementing a complex geometry into a proper, production rendering environment. Using Blender's Cycles physically based, ray-tracing rendering environment, a benchmark was produced by simulating a 3 meter by 3 meter laboratory room, with standard white walls. The design selected for rendering was based on a triply periodic minimal surface (i.e., Schwarz Gyroid). It was placed in the center of the room on a black floor to be tested under various lighting conditions for validation, as illustrated in FIG. 12. The camera in the simulation was a low field-of-view camera placed close to the scaffold to be similar to the camera module mounted on the BIO X 3D-printing system, which was used to photograph many of the fabricated bone scaffolds in this work. In the rendering environment, to match a complex, reflective, real-world material, a physically-based material was developed to model a white, glossy, reflective material similar to PCL's visual appearance. For validation of material invariance, a brown, rough physically-based material was generated as well; this is perhaps representing a wood-filled material or similar, however, it was selected to be in contrast to the glossy white material, to be used in comparison.

There were three different distinct lighting environments that were selected with the intention of: (a) creating variation in the visual appearance of the scaffold design, and (b) modeling possible different lighting environments that may be found in a standard lab or workspace setting. The first lighting environment tested in the simulated environment was the presence of a floor lamp with a white 50 W bulb, positioned close to a meter away from the scaffold, seen in the first row of FIG. 13. This offset light source causes a pronounced cast shadow in the deeper gradual indents of the pores, causing a perception of closer to nine pores on the surface. The next lighting environment was a large white fluorescent light panel in the ceiling of the room slightly offset, commonly found in laboratories, with the rendering shown in the second row of FIG. 13. And the third lighting environment simulated was the presence of a bright flashlight around half a meter directly above the sample. This also illuminated the gradual indents of the scaffold, but harsh reflected highlights can be seen produced as a result, this can be observed in the third row of FIG. 13. The final row of FIG. 13 displays the brown material variation in the 50 W lamp environment for comparison to glossy white plastic material.

The second column of FIG. 13 displays the visual pore highlighting results of the method described in this work. The color represents the size of an individual pore which was identified, with a marker representing the centroid of the pore, note that redder colors represent relatively larger pores and bluer colors represent relatively smaller pores.

The third column of FIG. 13 displays the efficacy of a standard methodology for identifying regions of porosity in recent scientific literature and industry. Specifically, the standard method used for a benchmark comparison is a method recently used for in situ monitoring of internal porosity using an optical camera for layer-by-layer material deposition. The method used functions on contrast thresholding, where the image is converted to grayscale, and a specific threshold is selected, where any pixel darker than a certain value is marked as porosity. The white marked regions can be observed in comparison to the individually highlighted regions in the second row. Note that the highlighted regions of the standard method of the third column all appear the same color and without markers, as the method used was for comparison of pore filling techniques. It is possible to run a simple component analysis to consolidate contacting pixels though.

To quantitatively demonstrate the robustness of the proposed method, Table 2 contains quantitative data scores for each of the four simulated environments in FIG. 13. The objective was to have similar quality metrics irrespective of lighting conditions, and any standard deviation demonstrates the quantified error due to variable lighting conditions of the algorithm.

TABLE 2
Measurements of comparison for the identical scaffold model, rendered
in different lighting conditions, to test the robustness of the
model. The “RSD Area” represents the percent relative
standard deviation (RSD) of pore areas, with “RSD Distance”
representing the percent RSD of pore pattern spacing distance,
and with the “Precision Error” being the weighted average of the two.

| | RSD Area | RSD Dist. | Precision |
|---|---|---|---|
| Environment | (%) | (%) | Error (%) |
| (a) 50 W Floor Lamp | 31.63 | 18.31 | 22.75 |
| (b) Fluorescent Ceiling Lighting | 43.50 | 11.64 | 22.26 |
| (c) Flash-Light (50 cm above) | 81.39 | 23.55 | 42.83 |
| (d) 50 W Floor Lamp, Clay Material | 9.16 | 1.51 | 4.06 |
| Standard (Std.) Deviation | 30.21 | 9.51 | 15.84 |

### Comparing the Presented Approach to the Standard Method.

Comparing the Accuracy of Pore Filling. In comparing the accuracy of the pore filling algorithms on the benchmarks, it was important to establish the objective of the pore filling algorithm, and the challenge with gradually forming pores. The challenge with identifying the porosity of a printed scaffold through only optical photographs, is that without the use of depth mapping, the only information that can be gathered on the scaffold is through reflected light. If the scaffold has discrete, sharp edges, then identifying the surface porosity is simple, however, with gradually forming smooth edges, as shown in FIG. 10, it is not trivial to define a point along the dropping curvature where a pore is defined; furthermore, identifying that same point along the curvature in dynamic lighting conditions is a complex challenge and beyond the scope and objectives of this work. The objective was not to identify the percent of the surface, which was marked as belonging to a pore, but to define quality metrics that measure the consistency of the porosity, which can be approximated in most lighting conditions (as discussed in the following sections).

In comparison to the “gold standard” technique for identifying porosity, thresholding techniques are the dominant technique due to their simplicity and efficacy. Testing the standard method on the rendered images of the scaffolds produced quality results across lighting conditions after being processed through the wisp cropping module defined in this work. The standard thresholding techniques function well with a well selected threshold with even lighting conditions, as depicted in the third column of FIG. 13. In comparison, the proposed method performed very similarly, except for the presence of some introduced noise due to the addition of all the processing steps.

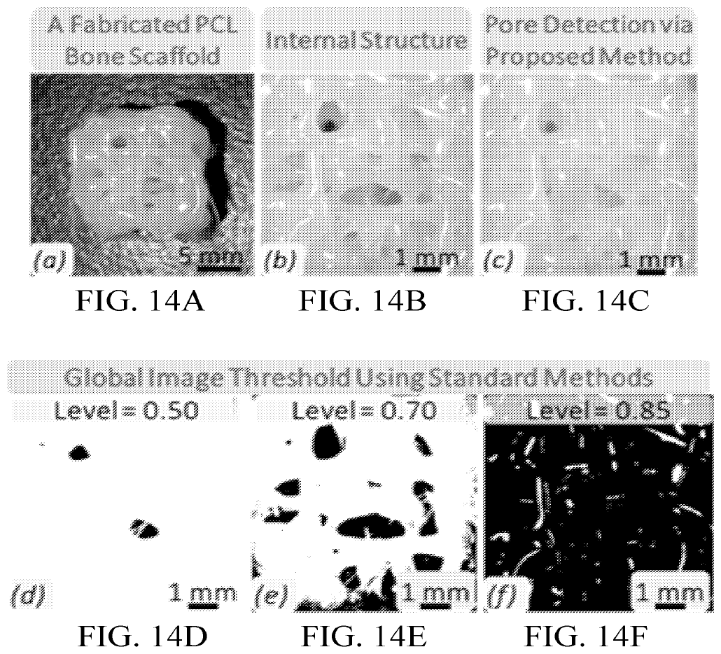

The feature-by-feature analysis for filling the pores until collision with the boundaries has the benefit of functioning automatically across lighting conditions, while traditional thresholding techniques are highly sensitive to the selected threshold for images without high contrast. FIG. 14(a)-(b) depicts a real-world print in PCL of the Schwarz gyroid scaffold used in the renderings. Due to challenges in printing PCL, the scaffold does not appear very similar to the rendering. The final three images, FIGS. 14(d), (e), and (f) demonstrate the sensitivity of small threshold changes to the resulting porosity labeling. The standard solution to this is to use automatic thresholding selection algorithms, such as Otsu's algorithm. However, Otsu's algorithm only works on bimodal intensity distributions, where there are clear dark regions and clear light regions in the image. Others had less challenge with this by using only diffusive materials, with a light directly horizontal to the surface to create sharp shadows, and because the pores that their work dealt with were sharply defined hemispherical crater defects. Otsu's algorithm fails to function on a multimodal image as shown in FIG. 14(b), as in real images, it is rare to see the dark base plate or deep pores that are seen in the rendering. Due to compiled errors, it is more common to see complex material pattern below the surface pores. Other algorithms, such as adaptive thresholding used for optical character recognition failed to function in testing on the large gradual pores. In complex real-world scenarios, the proposed method functioned far better in pore filling, as shown in FIG. 14(b), with seldom adjustment to program parameters.

In addition, a quantitative analysis was performed to further assess the performance of the proposed method. It was observed that the proposed method had an accuracy of 60.86% (defined as total accurate overlap with the real pore area−total falsely labeled area)/total real pore area), while the standard method (i.e., based on the global contrasting method) had an accuracy of 7.74%, −5.53%, and −534.26% for the manually selected threshold of 0.5, 0.7, and 0.85, respectively, as shown in FIG. 14.

Validation of Analysis Methods. In comparing data analysis methods to the standard method, the most common metrics are measurements such as the average area of porosity, standard deviation of pore size, centroid position identification, etc. An important difference of the proposed method is the preprocessing step to the data analysis, where pores which are not contacting are consolidated by estimating if different regions correspond to the same pore. This is in contrast to the standard “connected-component labeling” techniques used to simply group all contacting pixels. This method can be seen to be working effectively in the renderings in FIG. 13 (a-ii) and (d-ii) where even groups of pixels which are not contacting are successfully grouped into corresponding to the same pore, however, the model does introduce noise in very specific conditions, such as FIG. 13 (c-ii), where it can be observed that portions of the bottom three pores were all consolidated into one scaffold (as shown in red for its size). This module of the proposed method is very effective in improving consistency of results in messy, real world scaffolds, however, the pore in FIG. 13 (c-ii) was consolidated due to more subtle shadowed connections between the pores in the rendering, shown in FIG. 13 (c-i).

In reviewing the data quantification metrics in Table 2, comparing the quality scores of the different scaffolds rendered in different lighting conditions, it can be seen that the issues related to using a rendering for a benchmark, and the difficulty of the benchmark used, caused minor errors in the pore filling and consolidation algorithm, which led to the introduction of major noise into the quality results. Referring to FIG. 13, first, when observing the noise present in FIG. 13 (a-ii), the algorithm decided that the hole pairs belonged to the same pore, as they were present in the same surface indentation, in eight out of the nine indentations, with breaking the 9^{th} pore up into two smaller pores. This resulted in a high RSD of Areas, and RSD of Distances. On the next environment, in FIG. 13 (b-ii), the algorithm opted to break up each pore except for one, creating the same issue. In FIG. 13 (c-ii), the pores were all separate, except for one additional ridge line pore, and the major error of consolidating the bottom three pores. And only in the last environment with the diffuse material, did the algorithm maintain consistency in consolidation, as seen in FIG. 13 (d-ii). As a result, over the four environments, the fourth environment gave the correct answer, with the RSD of distance being close to zero at 1.51%. The precision error, the weighted average of both metrics with a bias towards RSD of distance had a final standard deviation of 15.84 due to the noise in variation of widely varying lighting conditions in a rendering environment. Note that it is challenging to achieve substantial lighting varying to the extent demonstrated in a lab environment, as there is always a combination of lights illuminated the room, with more surfaces to diffuse of, as compared to the simulation environment where the light sources shown were the only sources present. Interestingly, the pore fillings appear near identical in FIG. 13 (a-ii) and FIG. 13 (d-ii) so it would appear that the failure to consolidate one of the pores correctly in FIG. 13 (a-ii) is an outlier, as discussed below.

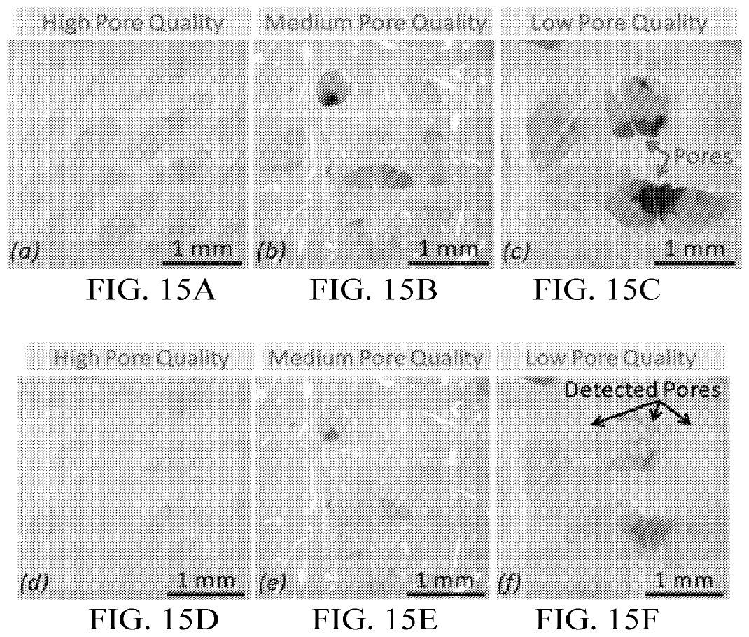

Comparing Quality Estimates of the Proposed Method to Compression Testing Metrics. In the prior work, multiple scaffold designs were selected for printing and analysis of mechanical properties. With printing of multiple samples of each design, the compression modulus of each of the samples was tested in a compression testing apparatus. For this work, before the samples were destroyed (compressed), surface images were collected for three of the scaffold designs, demonstrated in FIG. 15 (a)-(c).

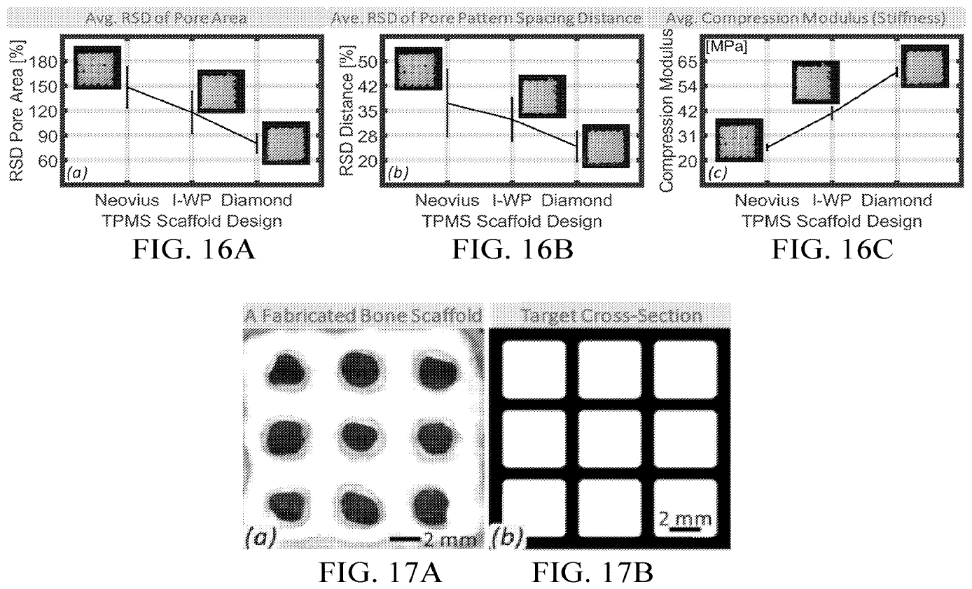

As demonstrated in FIG. 15, there was a discernable variation in manufacturing quality for the three scaffold designs, however, in FIG. 15 (d-f), the proposed method was able to function well on the inconsistent scaffolds. The average quality metrics for the three scaffold designs are displayed in FIGS. 16 (a) and (b); it can be observed that under real world conditions, the standard deviation for metrics on the scaffold sets was lower than that of the renderings.

Referring to FIG. 16, it can be observed that, using the proposed method on a single photograph, in general lighting conditions, as the average quality scores of surface porosity improved (the error decreased), the average compression modulus of the PCL printed scaffolds increased. Observing the corresponding error bars which represent the standard deviation for the quality metrics, it can be observed that there is substantial variation between quality scores of the printed scaffolds. While there is actual variation between fabricated scaffolds, as can be observed in the standard deviation of compression modulus tests herein, there is significant noise in the proposed method's final quality measurements, which is likely the source of the substantially higher standard deviation in the quality measurements.

The model geometry utilized was designed for strength given a certain global density. If the printed scaffold deviates from the target geometry, it is expected that the strength of the scaffold will suffer, and the low and medium quality scaffolds in FIG. 15, which appear completely different from their target geometry patterns demonstrate such. Since each of the scaffold geometries meets the previously defined requirements for valid quality metrics, any error measured by the proposed method would result in a weaker printed scaffold, assuming no measurement noise was present.

The data collected was only from that of a single photo directed at the top of the printed scaffold, which disregards the internal structure of the scaffold which may affect the scaffold strength, but as seen in FIG. 16, there is some correspondence to just the surface quality and the compression modulus. This is perhaps a result of the PME process being sensitive to internal defects, where since new layers are printed on previous layers, and depend on the quality of previous layers, a defect significantly affects quality for many layers after, especially with a biomaterial such as PCL, which takes a long time to cool. Another explanation for this phenomenon could be a result of the compression strength being dependent on weakest load-bearing elements of the scaffold. And the weakest portion of the scaffold is the lowest quality portion, which is often the last layers, as demonstrated in FIG. 18.

In prior work, prints with identical process parameters and printer instructions would vary between prints due to variation in environmental factors and material factors; as a result, it is unknown if printed scaffolds met the same mechanical strength as previously printed and tested scaffolds, without testing the newly printed scaffold and destroying it. The proposed method demonstrates a rough analysis of comparing the material properties of a porous printed part, to that of one that was previously tested through a single photo, in general lighting conditions. The issue with this approach is the noise of surface porosity mappings, which can be improved in future works and mitigated with further results below, but more so, the issue with the proposed method in estimating quality for mechanical strength standards is the lack of information regarding the internal structure of the printed parts, which will be remedied in further results below.

Layer-by-Layer Analysis and an Extension of the Method with Target Model Awareness. The previous results were using analysis techniques general to any scaffold design which met the requirements, with the analysis simply checking consistency of porosity sizing and consistency of pattern spacing. However, a target model aware methodology opens the door for far more analysis to get more consistent results. It even allows for noise filtering, which drastically improves the consistency of results.

By taking the target scaffold geometry and using CAD software to take a cross-section at a specific layer, the exact target surface porosity at that layer can then be stored as a binary image. Performing this for every printed layer height, a library of binary images is generated for comparing the printed scaffold to the target geometry at any given height.

The first most significant advantage of this approach is that′ after optimally matching the generated reference image to the photograph of the scaffold, the reference image is used for substantial noise reduction in discarding minor pores or indentations which are likely not significant to the overall quality of the measurements. A depiction of this more robust approach is depicted in FIG. 17.

To demonstrate the efficacy of this approach, utilizing the 3D printer's camera module, a photo was automatically taken of the 3D print after each layer, with the analysis program receiving the photos and building a quality metric graph in real-time. Note that for layer-by-layer experiments, the material was changed to a starch-based bone scaffolding material which prints more quickly, and tends to deviate from target geometry more quickly, this was done for more rapid data collection.

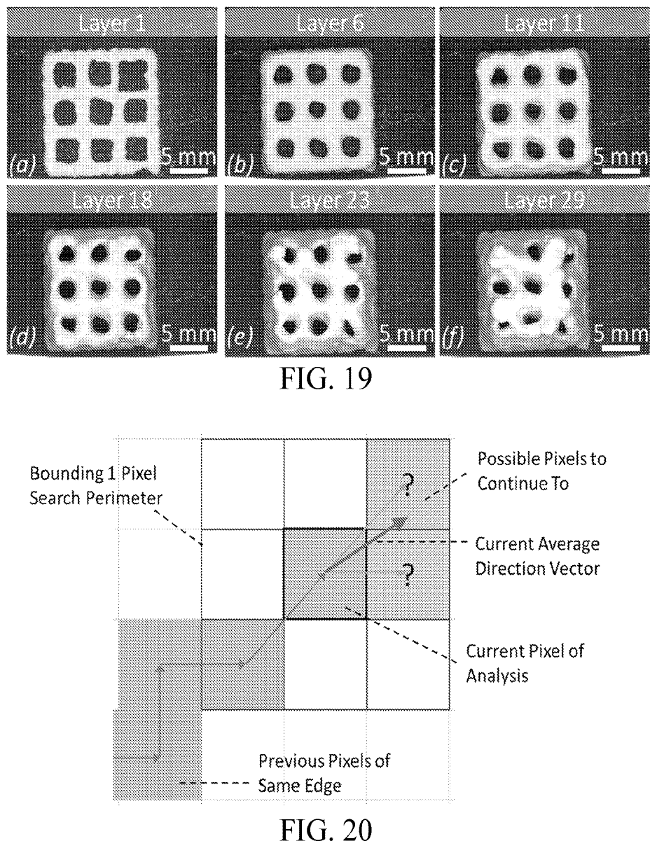

FIG. 18 depicts the quality metrics measured of a sample bone scaffold which had a gradual quality degradation as the print progressed. Observing the first 20 layers, the RSD-Area and RSD-Distance (pattern spacing consistency) metrics are holding very stable, degrading gradually as demonstrated in FIG. 19. This consistency of the ‘precision error’ metric exhibits the efficacy of the benefits of the model aware noise reduction, with the only major errors being on layer 21, when a pore was unrecognized, and on layer 24, when two pores were wrongly consolidated. The error spikes that follow are a result of actual error in the printed scaffold. These errors can easily be resolved in a future work which takes a model aware approach into consideration when initially performing the pore filling, as opposed to just at a final step where target geometry is only used for noise reduction.

Beyond using a model aware approach only for noise reduction and improving pore filling, by using a model aware approach, far more data metrics can be calculated by comparing to target geometry, with the metrics previously defined herein. As shown in FIG. 18, the metrics ‘% deviation from STL’ and ‘over-underflow %’ are calculated through a model aware comparison, and the two metrics depict much more consistent results. Because in the PME printing process, the quality of a surface layer is dependent on the quality of the layer which it is deposited on, it is expected that a curve depicting a quantified measure of print quality would not have sudden discontinuous jump in improvement (while a sudden jump in error is possible in the case of print errors). While the ‘precision error’ metrics are seen to have sudden improvements in quality, such as from layer 21 to 22, or from 25 to 26, which simply corresponds to noise caused by the distortions at the end of the print, the model-aware metrics maintain an impressive consistency and ability to filter noise, even under extreme distortions found at the end of the print, shown in FIG. 19.

The resulting final metric, titled ‘Overall Error’, is a weighted average of the ‘precision error’ metrics and the model-aware metric, utilizing all the previously collected data, and it represents a final calculated numerical quantification of quality for a singular layer of the print of the bone scaffold. Due to the wide array of data used and the improved metrics, the ‘overall error’ score is consistent and robust to noise. Earlier results in this work depicted a correlation between compressive strength of printed scaffolds and their single-image, noisy ‘precision error’ metrics, without any reference of the target geometry, so, it is expected that by utilizing the layer-by-layer curve for ‘overall error’, a much stronger correlation could be derived in future works.

### CONCLUSIONS

In summary, a novel image-based method was developed for the detection of complex gradual pores and subsequent quantification of the porosity of PME-fabricated bone tissue scaffolds. The presented method is unique in the sense that it is capable of (i) detecting individual pores of varying geometry from a single camera image under a robust set of lighting conditions using a handcrafted image processing algorithm, and (ii) conducting a range of porosity analyses with or without knowledge from the target geometry of a scaffold. The proposed method allows for both labeling of scaffold pores and quantification of porosity on a layer-by-layer basis, used for the non-destructive assessment of the functional performance of fabricated bone tissue scaffolds.

It was observed that, while the algorithm was able to robustly adjust pore-filling parameters in different lighting conditions, the noise from varying lighting conditions had an impact on the quality metrics (when lacking the model-aware noise rejection analysis). In addition, in comparison to the standard approach used in recent literature, tested on the ideal scaffold rendering, after manually adjusting the threshold values, the standard approach performed comparably in filling pores to the pore filling of the proposed method; however, in real world testing, the proposed method performed substantially better in pore filling and consolidation than the standard method.

To validate the applicability of the proposed method, a relationship was demonstrated between the quality scores from a single top layer photo of fabricated scaffolds and their corresponding compressive strength, using model-unaware metrics, which led to substantial noise present in the results. However, the noise was substantially reduced by referencing binary image cross-sections of the target geometry along with new quality metrics (utilizing the target geometry cross-sections). Overall, by combining the model-aware and general quality metrics, the noise was near entirely eliminated.

With the noise-free layer-by-layer quality assessment, it is expected that a much stronger relationship can be derived, comparing the compressive strength of the fabricated bone scaffolds to the quantified quality error curves/trends. Also, the proposed method allows for a real-time comparison between layer-by-layer fabricated scaffold quality and that of a reference scaffold (meeting a target strength/stiffness) and thus is capable of verifying whether newly fabricated scaffolds meet quality standards or not. The results of the proposed method are of significant value in a clinical environment (as well as in other fields highly sensitive to print quality, e.g., aerospace), as there is a need to validate the strength of fabricated scaffolds (having a complex internal structure) prior to implantation into a patient. Based on the model-aware approach and enhanced noise reduction, the resulting layer-by-layer pore fillings can also be used to create a rough, approximate 3D model of the scaffolds for analysis of other parameters, such as estimating overall volumetric porosity. Last but not least, the presently-described method is of great value in characterizing and monitoring changes/drifts in the quality of fabricated scaffolds for the optimization of the PME process.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Giannoudis, P. V., Faour, O., Goff, T., Kanakaris, N., and Dimitriou, R., 2011, “Masquelet Technique for the Treatment of Bone Defects: Tips-Tricks and Future Directions,” Injury, 42(6), pp. 591-598, DOI: 10.1016/j.injury.2011.03.036.
2. Pedrero, S. G., Llamas-Sillero, P., and Serrano-López, J., 2021, “A Multidisciplinary Journey Towards Bone Tissue Engineering,” Materials, 14(17), p. 4896, DOI: 10.3390/ma14174896.
3. Sun, Z., Wu, F., Gao, H., Cui, K., Xian, M., Zhong, J., Tian, Y., Fan, S., and Wu, G., 2020, “A Dexamethasone-Eluting Porous Scaffold for Bone Regeneration Fabricated by Selective Laser Sintering,” ACS Applied Bio Materials, 3(12), pp. 8739-8747, DOI: 10.1021/acsabm.0c01126.
4. Thadavirul, N., Pavasant, P., and Supaphol, P., 2014, “Development of Polycaprolactone Porous Scaffolds by Combining Solvent Casting, Particulate Leaching, and Polymer Leaching Techniques for Bone Tissue Engineering,” Journal of Biomedical Materials Research Part A, 102(10), pp. 3379-3392, DOI: 10.1002/jbm.a.35010.
5. Yu, M., Yeow, Y. J., Lawrence, L., Claudio, P. P., Day, J. B., and Salary, R., 2021, “Characterization of the Functional Properties of PCL Bone Scaffolds Fabricated Using Pneumatic Microextrusion” Journal of Micro- and Nano-Manufacturing, JMNM-20-1074, DOI: 10.1115/1.4051631.
6. Murphy, C. M., and O'Brien, F. J., 2010, “Understanding the Effect of Mean Pore Size on Cell Activity in Collagen-Glycosaminoglycan Scaffolds,” Cell adhesion & migration, 4(3), pp. 377-381, DOI: 10.4161/cam.4.3.11747.
7. Karageorgiou, V., and Kaplan, D., 2005, “Porosity of 3D Biomaterial Scaffolds and Osteogenesis,” Biomaterials, 26(27), pp. 5474-5491, DOI: 10.1016/j.biomaterials.2005.02.002.
8. O'Brien, F. J., Harley, B., Yannas, I. V., and Gibson, L. J., 2005, “The Effect of Pore Size on Cell Adhesion in Collagen-GAG Scaffolds,” Biomaterials, 26(4), pp. 433-441, DOI: 10.1016/j.biomaterials.2004.02.052.
9. Baldwin, C. A., Sederman, A. J., Mantle, M. D., Alexander, P., and Gladden, L. F., 1996, “Determination and Characterization of the Structure of a Pore Space from 3D Volume Images,” Journal of colloid and interface science, 181(1), pp. 79-92, DOI: 10.1006/jcis.1996.0358.
10. e Silva, R., Negri, R., and de Mattos Vidal, D., 2019, “A New Image-Based Technique for Measuring Pore Size Distribution of Nonwoven Geotextiles,” Geosynthetics International, 26(3), pp. 261-272, DOI: 10.1680/jgein.19.00005.
11. Chukwuma, K., Bordy, E. M., and Coetzer, A., 2018, “Evolution of Porosity and Pore Geometry in the Permian Whitehill Formation of South Africa-A FE-SEM Image Analysis Study,” Marine and Petroleum Geology, 91, pp. 262-278, DOI: 10.1016/j.marpetgeo.2017.12.026.
12. Chen, Z., Song, Y., Jiang, Z., Liu, S., Li, Z., Shi, D., Yang, W., Yang, Y., Song, J., and Gao, F., 2019, “Identification of Oganic Matter Components and Organic Pore Characteristics of Marine Shale: A Case Study of Wufeng-Longmaxi Shale in Southern Sichuan Basin, China,” Marine and Petroleum Geology, 109, pp. 56-69, DOI: 10.1016/j.marpetgeo.2019.06.002.
13. Kim, F., Moylan, S., Garboczi, E., and Slotwinski, J., 2017, “Investigation of Pore Structure in Cobalt Chrome Additively Manufactured Parts Using X-Ray Computed Tomography and Three-Dimensional Image Analysis,” Additive Manufacturing, 17, pp. 23-38, DOI: 10.1016/j.addma.2017.06.011.
14. Du Plessis, A., Sperling, P., Beerlink, A., Tshabalala, L., Hoosain, S., Mathe, N., and Le Roux, S. G., 2018, “Standard Method for MicroCT-Based Additive Manufacturing Quality Control 1: Porosity Analysis,” MethodsX, 5, pp. 1102-1110, DOI: 10.1016/j.mex.2018.09.005.
15. Leonard, F., Tammas-Williams, S., and Todd, I., “CT for Additive Manufacturing Process Characterisation: Assessment of melt strategies on defect population,” Proc. 6th Conf. on Industrial Computed Tomography (ICT 2016), 9-12 Feb. 2016, Wels, Austria, pp. 1-8, DOI:
16. Villarraga, H., Lee, C., Corbett, T., Tarbutton, J. A., and Smith, S. T., “Assessing Additive Manufacturing Processes with X-Ray CT Metrology,” Proc. ASPE Spring Topical Meeting: Achieving Precision Tolerances in Additive Manufacturing, North Carolina State University, Raleigh, USA, 26-29 Apr. 2015., ASPE, pp. 116-121, DOI:
17. Holzmond, O., and Li, X., 2017, “In Situ Real Time Defect Detection of 3D Printed Parts,” Additive Manufacturing, 17, pp. 135-142, DOI: 10.1016/j.addma.2017.08.003.
18. Aminzadeh, M., 2016, “A Machine Vision System for In-Situ Quality Inspection in Metal Powder-Bed Additive Manufacturing,” Doctoral Dissertation, Advisor: Dr. Thomas Kurfess, Georgia Institute of Technology, Atlanta, GA, USA., DOI: http://hdl.handle.net/1853/56291.
19. Hu, D., and Kovacevic, R., 2003, “Sensing, Modeling and Control for Laser-Based Additive Manufacturing,” International Journal of Machine Tools and Manufacture, 43(1), pp. 51-60, DOI: 10.1016/S0890-6955(02)00163-3.
20. Krauss, H., Eschey, C., and Zaeh, M., “Thermography for Monitoring the Selective Laser Melting Process,” Proc. 2012 International Solid Freeform Fabrication Symposium, The University of Texas at Austin, TX, USA, Aug. 22 2012., University of Texas Libraries, DOI: 10.26153/tsw/15406.
21. Craeghs, T., Clijsters, S., Kruth, J.-P., Bechmann, F., and Ebert, M.-C., 2012, “Detection of Process Failures in Layerwise Laser Melting with Optical Process Monitoring,” Physics Procedia, 39, pp. 753-759, DOI: 10.1016/j.phpro.2012.10.097.
22. Bowoto, O. K., Oladapo, B. I., Zahedi, S., Omigbodun, F. T., and Emenuvwe, O. P., 2020, “Analytical Modelling of In Situ Layer-Wise Defect Detection in 3D-Printed Parts: Additive Manufacturing,” The International Journal of Advanced Manufacturing Technology, 111(7), pp. 2311-2321, DOI: 10.1007/s00170-020-06241-6.
23. Beucher, S., “Use of Watersheds in Contour Detection,” Proc. Proceedings of the International Workshop on Image Processing: Real Time Edge and Motion Detection/Estimation, Rennes, France, Sep. 17-21, 1979, CCETT, DOI: N/A.
24. Otsu, N., 1979, “A Threshold Selection Method from Gray-Level Histograms,” IEEE Transactions on Systems, Man, and Cybernetics, 9(1), pp. 62-66, DOI: 10.1109/TSMC.1979.4310076.
25. Bradley, D., and Roth, G., 2007, “Adaptive Thresholding using the Integral Image,” Journal of Graphics Tools, 12(2), pp. 13-21, DOI: 10.1080/2151237X.2007.10129236.
26. Zhao, D., Yu, M., Lawrence, L., Claudio, P. P., Day, J. B., and Salary, R. R., “Investigation of the Influence of Consequential Design Parameters on the Mechanical Performance of Biodegradable Bone Scaffolds, Fabricated Using Pneumatic Micro-Extrusion Additive Manufacturing Process,” Proc. Paper No. 8512, ASME 2020 International Manufacturing Science and Engineering Conference (MSEC 2020), Cincinnati, OH, USA, Jun. 22-26, 2020., American Society of Mechanical Engineers, DOI: 10.1115/MSEC2020-8512.
27. Yeow, Y. J., Yu, M., Day, J. B., and Salary, R. R., “A Computational Fluid Dynamics (CFD) Study of Material Flow in Pneumatic MicroExtrusion (PME) Additive Manufacturing Process,” Proc. Paper No. 24325, ASME 2020 International Mechanical Engineering Congress and Exposition (IMECE2020), Portland, OR, USA, Nov. 16-19, 2020., American Society of Mechanical Engineers, DOI: 10.1115/IMECE2020-24325.
28. Salary, R., Lombardi, J. P., Weerawarne, D. L., Rao, P. K., and Poliks, M. D., “A Computational Fluid Dynamics (CFD) Study of Material Transport and Deposition in Aerosol Jet Printing (AJP) Process,” Proc. Paper No. 87647, ASME 2018 International Mechanical Engineering Congress & Exposition (IMECE 2018), Pittsburgh, PA, USA, Nov. 9-15, 2018., American Society of Mechanical Engineers, DOI: 10.1115/IMECE2018-87647.
29. Salary, R., Lombardi, J. P., Weerawarne, D. L., Rao, P., and Poliks, M. D., 2021, “A Computational Fluid Dynamics Investigation of Pneumatic Atomization, Aerosol Transport, and Deposition in Aerosol Jet Printing Process,” Journal of Micro- and Nano-Manufacturing, 9(1), p. 010903, DOI: 10.1115/1.4049958.
30. Salary, R., Lombardi, J. P., Tootooni, M. S., Donovan, R., Rao, P. K., Borgesen, P., and Poliks, M. D., 2017, “Computational Fluid Dynamics Modeling and Online Monitoring of Aerosol Jet Printing Process,” Journal of Manufacturing Science and Engineering, 139(2), p. 021015, DOI: 10.1115/1.4034591.
31. Spoerk, M., Gonzalez-Gutierrez, J., Sapkota, J., Schuschnigg, S., and Holzer, C., 2018, “Effect of the Printing Bed Temperature on the Adhesion of Parts Produced by Fused Filament Fabrication,” Plastics, Rubber and Composites, 47(1), pp. 17-24, DOI: 10.1080/14658011.2017.1399531.
32. Jansen, E. J., Sladek, R. E., Bahar, H., Yaffe, A., Gijbels, M. J., Kuijer, R., Bulstra, S. K., Guldemond, N. A., Binderman, I., and Koole, L. H., 2005, “Hydrophobicity as a Design Criterion for Polymer Scaffolds in Bone Tissue Engineering,” Biomaterials, 26(21), pp. 4423-4431, DOI: 10.1016/j.biomaterials.2004.11.011.
33. Jenkins, M., and Stamboulis, A., 2012, Durability and Reliability of Medical Polymers, Woodhead Publishing, Aug. 13, 2012, Philadelphia, PA, USA, DOI: N/A.
34. Brakke, K., 2020, “Triply Periodic Minimal Surfaces,” Department of Mathematics and Computer Science, Susquehanna University, Selinsgrove, PA, USA.
35. Schoen, A. H., May 1970, Infinite Periodic Minimal Surfaces without Self-Intersections, National Aeronautics and Space Administration (NASA), Technical Note (NASA TN D-5541), Washington, DC, USA, DOI:
36. Piper, S., 2018, “Maths Models: Triply Periodic Minimal Surface Structures Mega Pack,” MyMiniFactory, London UK.
37. Klemstine, C., Abdelgaber, Y., Lawrence, L., Day, J. B., Claudio, P. P., and Salary, R. R., “Characterization of the Compressive Properties of Triply Periodic Minimal Surface PCL Scaffolds for Bone Tissue Engineering,” Proc. Paper No. IMECE2021-72125, ASME International Mechanical Engineering Congress & Exposition (IMECE 2021), Virtual Conference, Nov. 1-5, 2021., American Society of Mechanical Engineers, DOI: 10.1115/IMECE2021-72125.
38. Hoffman, D., Hoffman, J., Weber, M., Trazet, M., Wohlgemuth, M., Boix, E., Callahan, M., Thayer, E., and Wei, F., 2019, “The Scientific Graphics Project—Triply Periodic Level Surfaces,” Mathematical Sciences Research Institute, Berkeley, CA, USA.
39. Chaffins, A., Yu, M., Claudio, P. P., Day, J. B., and Salary, R. R., “Investigation of the Functional Properties of Additively-Fabricated Triply Periodic Minimal Surface-Based Bone Scaffolds for the Treatment of Osseous Fractures,” Proc. Paper No. 2004, ASME 2021 International Manufacturing Science and Engineering Conference (MSEC 2021), Virtual Conference (Due to the COVID-19 Pandemic), Hosted by the University of Cincinnati, Cincinnati, OH, USA, Jun. 21-25, 2021., American Society of Mechanical Engineers, DOI: 10.1115/MSEC2021-63413.
40. Salary, R., 2022, “Advanced Manufacturing for Bone Tissue Engineering and Regenerative Medicine,” IntechOpen, London, UK, p. 304, DOI: 10.5772/intechopen.102563.
41. Yu, M., Yeow, Y. J., Lawrence, L., Claudio, P. P., Day, J. B., and Salary, R. R., “Investigation of the Effects of Design and Process Parameters on the Mechanical Properties of Biodegradable Bone Scaffolds, Fabricated Using Pneumatic Microextrusion Process,” Proc. Paper No. 24252, ASME 2020 International Mechanical Engineering Congress and Exposition (IMECE2020), Portland, OR, USA, Nov. 16-19, 2020., American Society of Mechanical Engineers, DOI: 10.1115/IMECE2020-24252.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method for characterizing a bone tissue scaffold, comprising:
providing a bone tissue scaffold produced by an additive manufacturing process;
capturing an image of the bone tissue scaffold;
determining a boundary of the bone tissue scaffold included in the image;
detecting one or more surface features of the bone tissue scaffold using an edge detection algorithm, the one or more surface features including one or more pores and/or one or more highlights;
identifying edges that correspond to a pore using a pore filling algorithm; and
mapping the surface porosity of the bone tissue scaffold.

2. The method of claim 1, wherein the additive manufacturing method is pneumatic micro-extrusion.

3. The method of claim 1, wherein the bone tissue scaffold comprises a polyester polymer.

4. The method of claim 1, wherein capturing an image of the bone tissue scaffold comprises capturing an image of the bone tissue scaffold using a high-resolution charge-coupled device (CCD) camera.

5. The method of claim 4, wherein the image is a monochromatic image.

6. The method of claim 1, wherein determining a boundary of the bone tissue scaffold in the image comprises the following steps (a) to (d):
(a) positioning a bounding box around the bone tissue scaffold included in the image,
(b) incrementally shrinking the bounding box,
(c) monitoring an amount of material discarded each time the bounding box is shrunk; and
(d) identifying a rate of change of discarded material as compared to a predetermined threshold parameter, such that the boundary is determined when the rate of change exceeds the predetermined threshold parameter.

7. The method of claim 6, further comprising rotating the image of bone tissue scaffold and repeating steps (a) to (d) using the rotated image.

8. The method of claim 1, wherein using an edge detection algorithm to detect one or more surface features comprises using a Canny edge detection algorithm.

9. The method of claim 8, further comprising a step of identifying a continuous edge of the one or more detected surface features using a marching algorithm.

10. The method of claim 1, further comprising a step of filtering out edges corresponding to the one or more highlights prior to identifying edges corresponding to a pore.

11. The method of claim 10, wherein the step of filtering out edges corresponding to the one or more highlights comprises mapping a plurality of image intensities from the image, and calculating a gradient of the plurality of image intensities.

12. The method of claim 11, wherein the step of identifying edges corresponding to a pore using a pore filling algorithm comprises
identifying a pore edge from the mapping,
comparing the pore edge identified from the mapping to a corresponding pore in the image, the corresponding pore in the image identified using the edge detection algorithm, and
iteratively identifying an intersection of the pore edge from the mapping to the pore edge identified in the image.

13. A system for characterizing a bone tissue scaffold, comprising;
an image capture device for capturing an image of the bone tissue scaffold;
a processor for analyzing the image, the processor including instructions for
determining a boundary of the bone tissue scaffold shown in the image,
detecting one or more surface features of the bone tissue scaffold shown in the image using an edge detection algorithm, the one or more surface features including one or more pores and/or one or more reflections,
identifying edges that correspond to a pore shown in the image using a pore filling algorithm, and
mapping the surface porosity of the bone tissue scaffold shown in the image.

14. The system of claim 13, wherein determining a boundary of the bone tissue scaffold in the image comprises the following steps (a) to (d):
(a) positioning a bounding box around the bone tissue scaffold included in the image,
(b) incrementally shrinking the bounding box,
(c) monitoring an amount of material discarded each time the bounding box is shrunk; and
(d) identifying a rate of change of discarded material as compared to a predetermined threshold parameter, such that the boundary is determined when the rate of change exceeds the predetermined threshold parameter.

15. The system of claim 14, further comprising rotating the image of bone tissue scaffold and repeating steps (a) to (d) using the rotated image.

16. The system of claim 13, wherein using an edge detection algorithm to detect one or more surface features comprises using a Canny edge detection algorithm.

17. The system of claim 16, further comprising a step of identifying a continuous edge of the one or more detected surface features using a marching algorithm.

18. The system of claim 13, further comprising a step of filtering out edges corresponding to the one or more highlights prior to identifying edges corresponding to a pore.

19. The system of claim 18, wherein the step of filtering out edges corresponding to the one or more highlights comprises mapping a plurality of image intensities from the image, and calculating a gradient of the plurality of image intensities.

20. The system of claim 19, wherein the step of identifying edges corresponding to a pore using a pore filling algorithm comprises
identifying a pore edge from the mapping,
comparing the pore edge from the mapping to a corresponding pore in the image, the corresponding pore in the image identified using the edge detection algorithm, and
iteratively identifying an intersection of the pore edge from the mapping to the pore edge identified in the image.
